(19) United States
(12) Patent Application Publication
(10) Pub. No.: US 2025/0161204 A1
(43) Pub. Date: May 22, 2025

(54) COMPOSITIONS COMPRISING ULTRAFINE BUBBLES AND METHODS OF USING THEREOF IN BEVERAGES, FOOD PRODUCTS, SKIN AND HAIR CARE PRODUCTS, ORAL CARE PRODUCTS, FLAVOR AND SENSATION ENHANCERS, AND TOPICALLY APPLIED PRODUCTS

(71) Applicant: Hydrosome IP, LLC, Westmont, IL (US)

(72) Inventors: Peter A. Kozak, Bolingbrook, IL (US); John Nicholas Jackowetz, Rochester, NY (US); Carly S. Hanson, Naperville, IL (US); Kiriako D. Tsoukalas, Munster, IN (US); Bob Jacobs, Newtown, CT (US); David C. Bom, Union, KY (US)

(21) Appl. No.: 18/946,032

(22) Filed: Nov. 13, 2024

Related U.S. Application Data
(60) Provisional application No. 63/599,691, filed on Nov. 16, 2023.

(51) Int. Cl. A61K 9/00

(52) U.S. Cl. CPC A61K 9/0004; A61K 9/0014; A61K 9/0053; A61K 9/0095

(57) ABSTRACT

The disclosure provides compositions including ultrafine bubbles having water and gases released from solution in the water that dissolve, surround, and/or stabilize one or more non-gaseous solutes and/or gaseous solutes, as well as methods of making and using the compositions for consumer products and applications, such as beverages, oral care solutions, food products, skin care products, and hair care products. The methods of making the compositions including ultrafine bubbles include processes for dissolving, surrounding, and/or stabilizing non-gaseous solutes and/or gaseous solutes with ultrafine bubbles.

### FIELD

The present disclosure generally relates to aqueous compositions that comprise soft cavitated water and/or ultrafine bubbles and, optionally, non-gaseous solutes and/or gaseous solutes for use in beverages, skin and hair care products, oral care products, flavor and sensation enhancers, topically applied products, and other consumer products.

### BACKGROUND

Ultrafine bubbles in aqueous compositions can be generated when “cavitation” occurs. There are two fundamental types of cavitation: (1) vaporous or “hard” cavitation and (2) gaseous or “soft” cavitation. Vaporous cavitation occurs when the pressure in a liquid drops below the vapor pressure of the liquid, resulting in the formation of unstable low-pressure voids or bubbles formed from vaporized particles or molecules of the liquid itself. By contrast, gaseous cavitation occurs when gases dissolved within a liquid fall out of solution with decreasing pressure, typically at pressures higher than the vapor pressure of the liquid itself-creating bubbles formed from particles or molecules of the liquid and the released gases.

Typical aqueous ultrafine bubbles are created as a result of vaporous or “hard” cavitation, when the resulting voids collapse and form shockwaves. The shockwaves facilitate the formation of ultrafine bubbles from exogenous non-dissolved gases. These ultrafine bubbles have substantially different structural, functional, and stability characteristics than ultrafine bubbles formed from “gaseous” cavitation. In aqueous compositions, ultrafine bubbles formed from “hard” cavitation (which comprise bubbles including water molecules surrounding exogenously provided gases) have substantially different physical characteristics in terms of structure, function, and stability than ultrafine bubbles formed from “soft” cavitation (which comprise bubbles including water molecules surrounding gases released from solution within the water).

With respect to aqueous compositions, it is well known that the organization of water molecules influences the stability, solubility, and bioavailability of any solutes dissolved within. Such organization is also known to influence the bioavailability of the water itself. As the organization of water molecules in compositions having ultrafine bubbles produced via vaporous or “hard” cavitation differs substantially from that of the organization of water molecules in compositions containing ultrafine bubbles produced via gaseous or “soft” cavitation, the stability, solubility, and bioavailability of solutes dissolved within the two water compositions may be substantially different, as well as the bioavailability of the water molecules themselves. It would be beneficial to produce aqueous compositions including water and ultrafine bubbles comprising gases released from solution in water-that is, produced via gaseous or “soft” cavitation-which compositions have improved stability, solubility, and bioavailability as compared to compositions including no ultrafine bubbles or solutions comprising or consisting of aqueous ultrafine bubbles formed via vaporous or “hard” cavitation. There also exists a need for compositions that comprise water, ultrafine bubbles comprising gases released from solution in water, and a non-gaseous solute and/or gaseous solute that have improved bioavailability, solubility, and/or stability. The non-gaseous solutes and/or gaseous solutes with improved bioavailability have been shown to be effective in permeating cell membranes and delivering the non-gaseous solutes and/or gaseous solutes into cells. This ability to penetrate allows the compositions to be effective means to deliver bioactive agents and/or hydration into human cells.

Consumer products (such as beverages, oral care solutions, and over-the-counter skin care products) are often used to deliver bioactive agents and/or hydration to subjects. However, the efficacy and/or delivery of such bioactives and/or hydration is often limited. Thus, it would be helpful to develop compositions and methods that improve the efficacy and/or delivery of bioactives and/or hydration to subjects via easily obtained consumer products.

### SUMMARY

The present disclosure provides aqueous compositions and methods for improving the bioactive efficacy and/or hydration capabilities of consumer products (e.g., beverages, oral care products, skin and hair care products), the compositions or solutions including gaseous ultrafine bubbles (i.e., ultrafine bubbles that comprise or consist essentially of water and gases released from solution in the water and produced via gaseous or “soft” cavitation) and at least one non-gaseous solute and/or at least one gaseous solute dissolved within, surrounded by, and/or stabilized by the ultrafine bubbles.

In one aspect of the invention, the compositions are aqueous compositions for oral administration (e.g., mouthwashes) and/or ingestion (e.g., beverages). The compositions include water and ultrafine bubbles comprising gases released from solution in the water. In some embodiments, the composition increases cell permeability and/or bioavailability of the water within the composition. In some embodiments, the compositions include one or more non-gaseous solutes and/or one or more gaseous solutes (e.g., flavoring agents, antibacterial agents, fluoride sources, nutrients, electrolytes, minerals, warming-sensation agents, and cooling-sensation agents).

In some embodiments, the water is selected from deionized (“DI”) water, ultrapure water, tap water, groundwater, surface water, and reverse osmosis water. In some embodiments, the water has a resistivity between about 17 to about 18.2 meg-ohm cm. In further embodiments, the water has a pH of between about 3 to about 7. In some embodiments, the water has an oxidative reduction potential of about −200 mV to about 800 mV.

In some embodiments, the aqueous compositions including ultrafine bubbles comprising or consisting essentially of water and gases released from solution in the water have improved bioavailability relative to naturally occurring water, and relative to compositions including ultrafine bubbles not formed via gaseous cavitation. In some embodiments, the ultrafine bubbles comprising or consisting essentially of water and gases released from solution in the water improve bioavailability of the water by about 10%, about 20%, about 30%, about 40%, about 50%, about 60%, about 70%, about 80%, or about 90% relative to naturally occurring water, and/or relative to compositions including ultrafine bubbles not formed via gaseous cavitation. In further embodiments, the ultrafine bubbles comprising or consisting essentially of water and dissolved/surrounded/stabilized solutes improve bioavailability of the water by about 1%, about 2%, about 3%, about 4%, about 5%, about 6%, about 7%, about 8%, or about 9% relative to naturally occurring water, and/or relative to compositions including ultrafine bubbles not formed via gaseous cavitation. In some embodiments, the at least one non-gaseous solute and/or at least one gaseous solute is dissolved within, surrounded by, and/or stabilized by the ultrafine bubbles. In some embodiments, the composition increases cell permeability and/or bioavailability of the at least one dissolved non-gaseous solute and/or at least one gaseous solute. In some embodiments, the at least one non-gaseous solute and/or at least one gaseous solute is present at a concentration of 0.1-30% by weight of the composition.

In some embodiments, the at least one non-gaseous solute and/or at least one gaseous solute is stable within the composition for at least 2 years.

In some embodiments, the ultrafine bubbles have a median diameter of between 2-400 nanometers. In some embodiments, the ultrafine bubbles remain stable within the composition for at least two years. In particular embodiments, the ultrafine bubbles remain stable within the composition for at least 2.5 years. In some embodiments, the ultrafine bubbles are concentrated within the composition via rotary evaporation and/or cross flow filtration. In particular embodiments, concentrated ultrafine bubbles are stable within the composition for at least 2 years.

In some embodiments, the compositions for oral administration and/or ingestion include at least one non-gaseous solute and/or at least one gaseous solute. In some embodiments, the at least one non-gaseous solute includes one or more electrolytes and minerals. In some embodiments, the one or more electrolytes and minerals comprises magnesium, sodium, potassium, chloride, sulfate, benzoate, bicarbonate, zinc, or combinations thereof. In some embodiments, the at least one gaseous solute may include one or more

In some embodiments, the composition is a beverage for ingestion. In some embodiments, the beverage is a juice, a still beverage, a carbonated beverage, an energy drink, an electrolyte drink, an alcoholic beverage, a mocktail, coffee, tea, or sweetened, flavored water.

In some embodiments, the beverage for ingestion includes one or more flavoring agents. In particular embodiments, the one or more flavoring agents comprise one or more salts. In certain embodiments, the one or more salts comprise at least one of magnesium chloride, calcium chloride, and sodium bicarbonate. In particular embodiments, the one or more flavoring agents is a pungency enhancer. In certain embodiments, the pungency enhancer includes one or more agents derived from black pepper including piperine, chavicine, isopiperine, isochavicine, dihydropiperine, and combinations thereof.

In some embodiments, the beverage for ingestion includes one or more cooling-sensation agents. In particular embodiments, the one or more cooling-sensation agents includes one or more of menthol, menthyl lactate, WS-3, WS-23, menthyl carboxamides, and combinations thereof.

In some embodiments, the beverage for ingestion includes one or more warming-sensation agents. In particular embodiments, the one or more warming-sensation agents includes one or more of capsaicin, camphor, eugenol, sanshools, and combinations thereof.

In some embodiments, the beverage for ingestion includes one or more nutrients. In particular embodiments, the one or more nutrients includes dietary collagen. In some embodiments, the at least one non-gaseous solute includes at least one of maltodextrin, vitamin c, gum acacia, niacinamide, monkfruit extract, and zinc sulfate.

In some embodiments, the beverage for ingestion comprises 5 wt % or less of ethanol.

In another aspect, a method for enhancing and/or extending duration of a cooling sensation imparted by a beverage for ingestion in accordance with the disclosure herein is provided. The method comprises ingesting the beverage comprising the ultrafine bubbles. In some embodiments, the cooling sensation imparted by the beverage composition is enhanced in comparison to beverages lacking the ultrafine bubbles. In some embodiments, the cooling sensation imparted by the beverage composition is extended in duration in comparison to beverages lacking the ultrafine bubbles.

In another aspect, a method of enhancing and/or simulating the presence of ethanol within a beverage composition is provided. The method comprises ingesting a beverage composition comprising 5 wt % or less of ethanol and ultrafine bubbles in accordance with the disclosure herein. In some embodiments, the presence of ethanol is enhanced and/or simulated by the ultrafine bubbles of the beverage composition imparting a cooling sensation to the tongue to simulate ethanol evaporation from a tongue. In some embodiments, the presence of ethanol is enhanced and/or simulated by the ultrafine bubbles of the beverage composition increasing the sensory effect of the pungency enhancer to simulate alcohol burn sensation. In particular embodiments, the alcohol burn sensation imparted by the beverage composition is enhanced in comparison to beverages lacking the ultrafine bubbles.

In another aspect, a method for enhancing and/or extending duration of a cooling sensation imparted by a beverage composition is provided. The method includes ingesting the beverage composition comprising the ultrafine bubbles in accordance with the disclosure herein and one or more cooling-sensation agents. In some embodiments, the cooling sensation imparted by the composition is enhanced in comparison to beverages lacking the ultrafine bubbles.

In another aspect, a method for enhancing and/or extending duration of a warming sensation imparted by a beverage composition is provided. The method includes ingesting the beverage composition comprising the ultrafine bubbles in accordance with the disclosure herein and one or more warming-sensation agents. In some embodiments, the warming sensation imparted by the composition is enhanced in comparison to beverages lacking the ultrafine bubbles.

In another aspect, a method for enhancing, increasing, and/or retaining hydration in a subject is provided. The method comprises ingesting a beverage composition comprising the ultrafine bubbles in accordance with the disclosure herein. In some embodiments, the subject is a mammal. In particular embodiments, the subject is a human. In some embodiments, the subject ingests the composition prior to physical exertion. In some embodiments, the subject ingests the composition during physical exertion. In some embodiments, the subject ingests the composition after physical exertion. In some embodiments, the subject experiences a greater decrease in blood serum osmolality after ingesting the composition as compared to after ingesting beverages lacking the ultrafine bubbles. In some embodiments, the subject experiences a decrease in blood serum osmolality for at least 2 hours post physical exertion. In some embodiments, the subject experiences a greater increase in total body water (TBW) after ingesting the composition as compared to ingesting beverages lacking the ultrafine bubbles. In some embodiments, the subject experiences an increase in total body water (TBW) for at least 2 hours post physical exertion. In some embodiments, the subject experiences a greater increase in intracellular water (ICW) after ingesting the composition as compared to ingesting beverages lacking the ultrafine bubbles. In some embodiments, the subject experiences an increase in intracellular water (ICW) for at least 2 hours post physical exertion. In some embodiments, the subject experiences a more rapid increase in intracellular water (ICW) after ingesting the composition as compared to ingesting beverages lacking the ultrafine bubbles.

In another aspect, a method for enhancing absorption of and/or bioavailability of dietary collagen in a subject is provided. The method comprises ingesting a beverage composition comprising dietary collagen as a non-gaseous solute (e.g., a nutrient) and the ultrafine bubbles in accordance with the disclosure herein. In some embodiments, the subject is a mammal. In particular embodiments, the subject is human. In some embodiments, the subject experiences a greater increase in skin resiliency and/or elasticity after ingesting the beverage composition as compared to ingesting beverages including the dietary collagen but lacking the ultrafine bubbles.

In some embodiments, the composition is an oral care solution for oral administration. In some embodiments, the oral care solution is a toothpaste or mouthwash.

In some embodiments, the oral care solution includes one or more cooling-sensation agents. In particular embodiments, the one or more cooling-sensation agents includes one or more of menthol, menthyl lactate, WS-3, WS-23, menthyl carboxamides, and combinations thereof.

In some embodiments, the oral care solution includes one or more fluoride sources. In some embodiments, the one or more fluoride sources includes sodium fluoride, stannous fluoride, acidulated phosphate fluoride, and combinations thereof.

In some embodiments, the oral care solution includes one or more antibacterial agents. In particular embodiments, the one or more antibacterial agents includes one or more of menthol, thymol, eucalyptol, methyl salicylate, and combinations thereof.

In another aspect, a method for enhancing and/or extending duration of a cooling sensation imparted by an oral care composition is provided. The method includes orally administering the oral care composition comprising the ultrafine bubbles in accordance with the disclosure herein and one or more cooling-sensation agents. In some embodiments, the cooling sensation imparted by the oral care composition is enhanced in comparison to oral care compositions including the one or more cooling-sensation agents but lacking the ultrafine bubbles.

In another aspect, a method for enhancing antibacterial efficacy imparted by an oral care composition is provided. The method comprises ingesting the oral care composition comprising the ultrafine bubbles in accordance with the disclosure herein and the one or more antibacterial agents. In some embodiments, the antibacterial efficacy imparted by the oral care composition is enhanced in comparison to oral care compositions including the one or more antibacterial agents but lacking the ultrafine bubbles.

In another aspect, a method for increasing the delivery of fluoride to a subject is provided. The method comprises orally administering an oral care composition including the ultrafine bubbles in accordance with the disclosure herein and one or more fluoride sources to the subject. In some embodiments, the one or more fluoride sources includes sodium fluoride, stannous fluoride, acidulated phosphate fluoride, and combinations thereof. In some embodiments, the subject is a mammal. In particular embodiments, the mammal is a human. In some embodiments, the delivery of fluoride to the subject imparted by the oral care composition comprising the ultrafine bubbles and one or more fluoride sources is increased in comparison to delivery of fluoride by compositions including the one or more fluoride sources but lacking the ultrafine bubbles.

In some embodiments, the composition is an ingestible food product. In some embodiments, the ingestible food product includes one or more flavoring agents as at least one non-gaseous solute and/or at least one gaseous solute. In some embodiments, the one or more flavoring agents is a pungency enhancer. In particular embodiments, the pungency enhancer includes one or more agents derived from black pepper including piperine, chavicine, isopiperine, isochavicine, dihydropiperine, and combinations thereof.

In another aspect, a method for increasing a pungent sensation imparted by one or more pungency enhancers in an ingestible food product is provided. The method includes ingesting the food product composition including the ultrafine bubbles in accordance with the disclosure herein and the one or more pungency enhancers. In particular embodiments, the pungency enhancer includes one or more agents derived from black pepper including piperine, chavicine, isopiperine, isochavicine, dihydropiperine, and combinations thereof. In some embodiments, the sensation of pungency is increased by the ultrafine bubbles of the food product composition in comparison to food compositions including the one or more pungency enhancers but lacking the ultrafine bubbles.

In another aspect of the invention, the compositions are aqueous compositions for topical application or use. The compositions include water and ultrafine bubbles comprising gases released from solution in the water.

In some embodiments, the water is selected from DI water, ultrapure water, tap water, groundwater, surface water, and reverse osmosis water. In some embodiments, the water has a resistivity between about 17 to about 18.2 meg-ohm cm. In further embodiments, the water has a pH of between about 3 to about 7. In some embodiments, the water has an oxidative reduction potential of about −200 mV to about 800 mV.

In some embodiments, the at least one non-gaseous solute and/or at least one gaseous solute is dissolved within, surrounded by, and/or stabilized by the ultrafine bubbles. In some embodiments, the composition increases cell permeability and/or bioavailability of the at least one dissolved non-gaseous solute and/or at least one gaseous solute. In some embodiments, the at least one non-gaseous solute and/or at least one gaseous solute is present at a concentration of 0.1-30% by weight of the composition.

In some embodiments, the at least one non-gaseous solute and/or at least one gaseous solute is stable within the composition for at least 2 years.

In some embodiments, the ultrafine bubbles have a median diameter of between 2-400 nanometers. In some embodiments, the ultrafine bubbles remain stable within the composition for at least two years. In particular embodiments, the ultrafine bubbles remain stable within the composition for at least 2.5 years. In some embodiments, the ultrafine bubbles are concentrated within the composition via rotary evaporation and/or cross flow filtration. In particular embodiments, concentrated ultrafine bubbles are stable within the composition for at least 2 years.

In some embodiments, the composition further comprises at least one non-gaseous solute and/or at least one gaseous solute. In some embodiments, the at least one non-gaseous solute and/or at least one gaseous solute comprises one or more cooling-sensation agents, pain relief agents, numbing agents, warming sensation agents, hair restorative agents, antihistamines, and anti-itch agents.

In some embodiments, the composition is a soap, liquid, cream, paste, gel, moisturizer, lotion, skincare product, shaving cream, face mask, medicated bandage and/or dressing, shampoo, and/or conditioner.

In some embodiments, the composition is part of a medicated bandage and/or dressing comprising one or more aqueous medicated solutions (i.e., solutions or gels containing active medicinal ingredients to aid in wound healing, prevent infection, or to provide other therapeutic benefits). In particular embodiments, the medicated bandage and/or dressing includes one or more aqueous medicated solutions including at least one of: hydrogel dressings for maintaining a moist wound environment; alginate dressings made from seaweed for wound healing; foam dressings infused with medication(s) for therapeutic benefits; silver-impregnated dressings with antimicrobial properties; iodine-infused dressings for antiseptic purposes; antibiotic-impregnated dressings for bacterial infection control; and phenytoin-infused dressings to promote wound healing; and collagen dressings combined with medication, particularly useful for chronic or slow-healing wounds.

In some embodiments, the composition is a skincare product. In some embodiments, the skincare product includes one or more of niacinamide, bakuchiol, retinol, and Everwhite. As used herein, Everwhite, produced by Sino Lion is a stable ascorbic acid derivative of the formula 3-O-ethyl ascorbic acid, typically used for whitening and anti-aging products. Everwhite has been shown to inhibit the activity of tyrosinase copper ions to effectively inhibit the formation of melanin. Further, Everwhite has been shown to inhibit the inflammation of the skin and improving skin color and elasticity.

In particular embodiments, the skincare product/topical composition comprises niacinamide.

In particular embodiments, the skincare product/topical composition comprises

bakuchiol.

In particular embodiments, the skincare product/topical composition comprises Everwhite.

In particular embodiments, the skincare product/topical composition comprises retinol.

In some embodiments, the compositions for topical application or use include at least one cooling-sensation agent as a non-gaseous solute. In some embodiments, the one or more cooling-sensation agents includes one or more of menthol, menthyl lactate, WS-3, WS-23, and menthyl carboxamides.

In some embodiments, the compositions for topical application or use include at least one warming-sensation agent as a non-gaseous solute. In some embodiments, the one or more warming-sensation agents includes one or more of capsaicin, camphor, eugenol, and sanshools.

In some embodiments, the compositions for topical application or use include one or more pain relief agents, numbing agents, and anti-itch agents as part of the non-gaseous solute and/or the gaseous solute. In particular embodiments, the non-gaseous solute and/or the gaseous solute includes one or more pain relief agents and numbing agents. In certain embodiments, the one or more pain relief agents and numbing agents include one or more of lidocaine, benzocaine, pramoxine, phenol, and methyl salicylate. In particular embodiments, the compositions for topical application or use include one or more anti-itch agents. In some embodiments, the one or more anti-itch agents including one or more of diphenhydramine, azelastine, olopatadine, ketotifen, and hydrocortisone.

In some embodiments, the composition for topical application or use includes one or more anti-acne agents. In other embodiments, the anti-acne agent is one or more of benzoyl peroxide, salicylic acid, retinoids, topical antibiotics (e.g. clindamycin, erythromycin), azelaic acid, sulfur, alpha hydroxy acids (AHAs), nicotinamide (niacinamide), dapsone, and tea tree oil.

In some embodiments, the composition for topical application or use is a hair loss prevention product and/or a hair restorative product. In some embodiments, the hair loss prevention product and/or a hair restorative product includes one or more hair restorative agents as part of the at least one non-gaseous solute and/or at least one gaseous solute. In particular embodiments, the one or more hair restorative agents include one or both of minoxidil and finasteride.

In another aspect, a method for increasing hydration of skin cells of a subject is provided. The method includes topically administering a skincare product or topical composition including ultrafine bubbles according to the disclosure herein to a subject's intact skin. In some embodiments, the skin cells to which the skincare product/topical composition is administered have greater hydration in comparison to skin cells to which compositions lacking the ultrafine bubbles are topically applied. In some embodiments, the skin cells to which the skincare product/topical composition is administered have increased hydration after administration of the skincare product for a period of at least 2 months. In some embodiments, the skin cells to which the skincare product/topical composition is administered have increased hydration after administration of the skincare product for a period of at least 18 months. In some embodiments, a viable epidermis layer of the subject's intact skin has a greater concentration of water after administration of the skincare product in comparison to a viable epidermis layer of intact skin to which compositions lacking the ultrafine bubbles are topically applied. In some embodiments, the viable epidermis layer of the intact skin to which the skincare product is administered has increased hydration for a period of at least 2 months after administration of the skincare product. In some embodiments, the viable epidermis layer of the intact skin to which the skincare product is administered has increased hydration for a period of at least 18 months after administration of the skincare product.

In another aspect, a method for increasing a barrier of water at a surface of a stratum corneum layer of intact skin is provided. The method comprises topically administering a skincare product or topical composition including ultrafine bubbles according to the disclosure herein to a subject's intact skin. In some embodiments, the surface of the stratum corneum layer of the subject's intact skin has a greater concentration of water in the barrier after administration of the skincare product/topical composition in comparison to a surface of a stratum corneum layer of intact skin to which compositions lacking the ultrafine bubbles are topically applied. In some embodiments, the barrier of water at the surface of the stratum corneum layer persists for a period of at least 2 months after administration of the skincare product. In some embodiments, the barrier of water at the surface of the stratum corneum layer persists for a period of at least 18 months after administration of the skincare product.

In another aspect, a method of increasing niacinamide absorption into skin cells of a subject is provided. The method comprises topically administering a skincare product or topical composition including ultrafine bubbles and niacinamide according to the disclosure herein to a subject's intact skin. In some embodiments, the niacinamide is absorbed into a stratum corneum layer of the intact skin of the subject. In particular embodiments, the stratum corneum layer of the subject's intact skin has a greater concentration of niacinamide after administration of the skincare product/topical composition in comparison to a stratum corneum layer of intact skin to which compositions comprising niacinamide but lacking the ultrafine bubbles are topically applied. In some embodiments, the stratum corneum layer of the intact skin to which the skincare product is administered has increased niacinamide content for a period of at least 2 months after administration of the skincare product. In some embodiments, the stratum corneum layer of the intact skin to which the skincare product is administered has increased niacinamide content for a period of at least 18 months after administration of the skincare product. In some embodiments, the niacinamide is absorbed into a viable epidermis layer of the intact skin of the subject. In some embodiments, the viable epidermis layer of the subject's intact skin has a greater concentration of niacinamide after administration of the skincare product in comparison to a viable epidermis layer of intact skin to which compositions comprising niacinamide but lacking the ultrafine bubbles are topically applied. In some embodiments, the viable epidermis layer of the intact skin to which the skincare product/topical composition is administered has increased niacinamide content for a period of at least 2 months after administration of the skincare product. In some embodiments, the viable epidermis layer of the intact skin to which the skincare product is administered has increased niacinamide content for a period of at least 18 months after administration of the skincare product/topical composition. In some embodiments, the skin cells of the intact skin to which the skincare product comprising niacinamide is administered have a greater concentration of absorbed niacinamide after administration of the skincare product/topical composition in comparison to skin cells to which compositions comprising niacinamide but lacking the ultrafine bubbles are topically applied.

In another aspect, a method of increasing a barrier of niacinamide at a surface of a stratum corneum layer of intact skin of a subject is provided. The method comprises topically administering a skincare product or topical composition including ultrafine bubbles and niacinamide according to the disclosure herein to a subject's intact skin. In some embodiments, the surface of the stratum corneum layer of the subject's intact skin has a greater concentration of niacinamide in the barrier after administration of the skincare product in comparison to a surface of a stratum corneum layer of intact skin to which compositions comprising niacinamide but lacking the ultrafine bubbles are topically applied. In some embodiments, the barrier of niacinamide at the surface of the stratum corneum layer persists for a period of at least 2 months after administration of the skincare product. In some embodiments, the barrier of niacinamide at the surface of the stratum corneum layer persists for a period of at least 18 months after administration of the skincare product.

In another aspect, a method of increasing bakuchiol absorption into skin cells of a subject is provided. The method comprises topically administering a skincare product or topical composition including ultrafine bubbles and bakuchiol according to the disclosure herein to a subject's intact skin.

In another aspect, a method of increasing a barrier of bakuchiol at a surface of a stratum corneum layer of intact skin of a subject is provided. The method comprises topically administering a skincare product or topical composition including ultrafine bubbles and bakuchiol according to the disclosure herein to a subject's intact skin. In some embodiments, the surface of the stratum corneum layer of the subject's intact skin has a greater concentration of bakuchiol in the barrier after administration of the skincare product in comparison to a surface of a stratum corneum layer of intact skin to which compositions comprising bakuchiol but lacking the ultrafine bubbles are topically applied. In some embodiments, the barrier of bakuchiol at the surface of the stratum corneum layer persists for a period of at least 2 months after administration of the skincare product. In some embodiments, the barrier of bakuchiol at the surface of the stratum corneum layer persists for a period of at least 18 months after administration of the skincare product/topical composition.

In another aspect, a method of increasing retinol absorption into skin cells of a subject is provided. The method comprises topically administering a skincare product or topical composition including ultrafine bubbles and retinol according to the disclosure herein to a subject's intact skin.

In another aspect, a method of increasing a barrier of retinol at a surface of a stratum corneum layer of intact skin of a subject is provided. The method comprises topically administering a skincare product or topical composition including ultrafine bubbles and retinol according to the disclosure herein to a subject's intact skin. In some embodiments, the surface of the stratum corneum layer of the subject's intact skin has a greater concentration of retinol in the barrier after administration of the skincare product in comparison to a surface of a stratum corneum layer of intact skin to which compositions comprising retinol but lacking the ultrafine bubbles are topically applied. In some embodiments, the barrier of retinol at the surface of the stratum corneum layer persists for a period of at least 2 months after administration of the skincare product. In some embodiments, the barrier of retinol at the surface of the stratum corneum layer persists for a period of at least 18 months after administration of the skincare product/topical composition.

In another aspect, a method of increasing Everwhite absorption into skin cells of a subject is provided. The method comprises topically administering a skincare product or topical composition including ultrafine bubbles and Everwhite according to the disclosure herein to a subject's intact skin. In some embodiments, the Everwhite is absorbed into a stratum corneum layer of the intact skin of the subject. In particular embodiments, the stratum corneum layer of the subject's intact skin has a greater concentration of Everwhite after administration of the skincare product/topical composition in comparison to a stratum corneum layer of intact skin to which compositions comprising Everwhite but lacking the ultrafine bubbles are topically applied. In some embodiments, the stratum corneum layer of the intact skin to which the skincare product is administered has increased Everwhite content for a period of at least 2 months after administration of the skincare product. In some embodiments, the stratum corneum layer of the intact skin to which the skincare product is administered has increased Everwhite content for a period of at least 18 months after administration of the skincare product.

In another aspect, a method of increasing a barrier of Everwhite at a surface of a stratum corneum layer of intact skin of a subject is provided. The method comprises topically administering a skincare product or topical composition including ultrafine bubbles and Everwhite according to the disclosure herein to a subject's intact skin. In some embodiments, the surface of the stratum corneum layer of the subject's intact skin has a greater concentration of Everwhite in the barrier after administration of the skincare product in comparison to a surface of a stratum corneum layer of intact skin to which compositions comprising Everwhite but lacking the ultrafine bubbles are topically applied. In some embodiments, the barrier of Everwhite at the surface of the stratum corneum layer persists for a period of at least 2 months after administration of the skincare product. In some embodiments, the barrier of Everwhite at the surface of the stratum corneum layer persists for a period of at least 18 months after administration of the skincare product.

In another aspect, a method for enhancing and/or extending duration of a cooling sensation imparted to skin of a subject is provided. The method comprises topically applying the skincare or topical composition including a cooling sensation agent and the ultrafine bubbles in accordance with the disclosure herein to intact skin of a subject. In some embodiments, the one or more cooling-sensation agents includes one or more of menthol, menthyl lactate, WS-3, WS-23, and menthyl carboxamides. In some embodiments, the cooling sensation imparted to the intact skin by the composition is enhanced and/or extended in duration after administration of the skincare product/topical composition in comparison to cooling sensations imparted to intact skin by compositions including the one or more cooling-sensation agents but lacking the ultrafine bubbles.

In another aspect, a method for enhancing and/or extending duration of a warming sensation imparted to skin of a subject is provided. The method comprises topically applying the skincare or topical composition including a warming sensation agent and the ultrafine bubbles in accordance with the disclosure herein to intact skin of a subject. In some embodiments, the warming-sensation agents includes one or more of capsaicin, camphor, eugenol, and sanshools. In some embodiments, the warming sensation imparted to the intact skin by the composition is enhanced and/or extended in duration after administration of the skincare product/topical composition in comparison to warming sensations imparted to intact skin by compositions including the one or more warming-sensation agents but lacking the ultrafine bubbles.

In another aspect, a method for increasing absorption of pain relief agents and/or numbing agents by skin cells of a subject is provided. The method includes topically applying a skincare product/topical composition comprising ultrafine bubbles in accordance with the disclosure herein and the one or more pain relief agents and/or numbing agents to intact skin of a subject. In some embodiments, the one or more pain relief agents and numbing agents include one or more of lidocaine, benzocaine, pramoxine, phenol, and methyl salicylate. In some embodiments, the absorption of the pain relief agents and/or numbing agents within the intact skin after administration of the composition is increased in comparison to absorption of the pain relief agents and/or numbing agents applied to intact skin by compositions including the one or more pain relief agents and/or numbing agents but lacking the ultrafine bubbles. In some embodiments, pain relief imparted by the pain relief agents and/or numbing agents within the composition is enhanced and/or extended in duration after administration of the composition in comparison to pain relief imparted to intact skin by compositions including the one or more pain relief agents and/or numbing agents but lacking the ultrafine bubbles.

In another aspect, a method for increasing absorption of anti-itch agents by skin cells of a subject is provided. The method includes topically applying a skincare product/topical composition comprising ultrafine bubbles in accordance with the disclosure herein and the one or more anti-itch agents to intact skin of a subject. In some embodiments, the one or more anti-itch agents include one or more of diphenhydramine, azelastine, olopatadine, ketotifen, and hydrocortisone. In some embodiments, the absorption of the anti-itch agents within the intact skin after administration of the composition is increased in comparison to absorption of the anti-itch agents applied to intact skin by compositions including the one or more anti-itch agents but lacking the ultrafine bubbles.

In another aspect, a method for increasing absorption of hair restorative agents by skin cells of a subject's skin tissue is provided. The method includes topically applying a skincare product/topical composition comprising ultrafine bubbles in accordance with the disclosure herein and one or more hair restorative agents to intact skin of a subject. In some embodiments, the one or more hair restorative agents includes at least one of minoxidil and finasteride. In some embodiments, the absorption of the hair restorative agents within the intact skin after administration of the composition is increased in comparison to absorption of the hair restorative agents applied to intact skin by compositions including the one or more hair restorative agents but lacking the ultrafine bubbles.

In another aspect, a method for improving acne control is provided. The method includes topically applying an anti-acne composition comprising ultrafine bubbles in accordance with the disclosure herein and one or more anti-acne agents to intact skin of a subject. In some embodiments, the one or more anti-acne agents includes at least one of benzoyl peroxide, salicylic acid, retinoids, topical antibiotics, clindamycin, erythromycin, azelaic acid, sulfur, alpha hydroxy acids, nicotinamide, dapsone, and tea tree oil. In some embodiments, the absorption of the anti-acne agents within the intact skin after administration of the composition is greater in comparison to absorption of the anti-acne agents applied to intact skin by compositions including the one or more anti-acne agents but lacking the ultrafine bubbles. In some embodiments, the control of acne by the anti-acne agents of the composition is enhanced and/or extended in duration after administration of the composition in comparison to control of acne to intact skin by compositions including the one or more anti-acne agents but lacking the ultrafine bubbles.

In another aspect, a method of making a water composition is provided. The method includes pumping water through a transfer pipe and a nozzle into a hollow cylinder. The nozzle is located at the proximal end of the hollow cylinder. The nozzle further includes an intake hole located at a proximal face of the nozzle connected to the transfer pipe, and one or more jet openings located at a distal face of the nozzle that open into a chamber defined by the hollow cylinder. The pumped water passing through the one or more jet openings creates a vortex of water in contact with an inner surface of the chamber. The process further includes allowing the water to exit the chamber defined by the hollow cylinder after the vortex of water is created. In some embodiments, the method further comprises adding at least one non-gaseous solute to the exited water.

In some embodiments, the at least one non-gaseous solute and/or at least one gaseous solute comprises one or more of cooling sensation agents, warming sensation agents, antibacterial agents, pain relief agents, numbing agents, hair restorative agents, anti-itch agents, topical antihistamines, pungency agents, skin hydration agents, flavoring agents, nutrients, electrolytes, minerals, alcohols, and fluoride sources. In some embodiments, the cooling sensation agents include at least one of menthol, menthyl lactate, WS-3, WS-23 and other menthyl carboxamides. In some embodiments, the warming sensation agents include at least one of capsaicin, camphor, eugenol, and sanshools. In some embodiments, the pain relief agents include at least one of lidocaine, benzocaine, pramoxine, phenol, and methyl salicylate. In some embodiments, the numbing agents include at least one of lidocaine, benzocaine, pramoxine, phenol, and methyl salicylate. In some embodiments, the hair restorative agents include at least one of minoxidil and finasteride. In some embodiments, the anti-itch agents include at least one of diphenhydramine, azelastine, olopatadine, ketotifen, and hydrocortisone. In some embodiments, the pungency agents include at least one of piperine, chavicine, isopiperine, and isochavicine. In some embodiments, the fluoride sources include at least one of sodium fluoride, stannous fluoride, and acidulated phosphate fluoride. In some embodiments, the one or more flavoring agents comprise one or more salts. In particular embodiments, the one or more salts comprise at least one of magnesium chloride, calcium chloride, and sodium bicarbonate. In some embodiments, the at least one non-gaseous solute comprises one or more electrolytes and minerals. In particular embodiments, the one or more electrolytes and minerals comprises magnesium, sodium, potassium, chloride, sulfate, benzoate, bicarbonate, zinc, or combinations thereof.

In another aspect, a method of making a water composition is provided. The method includes pumping water and at least one non-gaseous solute and/or at least one gaseous solute through a transfer pipe and a nozzle into a hollow cylinder. The nozzle is located at the proximal end of the hollow cylinder. The nozzle further includes an intake hole located at a proximal face of the nozzle connected to the transfer pipe, and one or more jet openings located at a distal face of the nozzle that open into a chamber defined by the hollow cylinder. The pumped water passing through the one or more jet openings creates a vortex of water in contact with an inner surface of the chamber. The process further includes allowing the water to exit the chamber defined by the hollow cylinder after the vortex of water is created. In some embodiments, the method further comprises adding at least one additional non-gaseous solute and/or at least one gaseous solute to the exited water.

In some embodiments, the at least one non-gaseous solute and/or at least one gaseous solute comprises one or more of cooling sensation agents, warming sensation agents, antibacterial agents, pain relief agents, numbing agents, hair restorative agents, anti-itch agents, topical antihistamines, pungency agents, skin hydration agents, flavoring agents, nutrients, electrolytes, minerals, alcohols, and fluoride sources. In some embodiments, the cooling sensation agents include at least one of menthol, menthyl lactate, WS-3, WS-23 and other menthyl carboxamides. In some embodiments, the warming sensation agents include at least one of capsaicin, camphor, eugenol, and sanshools. In some embodiments, the pain relief agents include at least one of lidocaine, benzocaine, pramoxine, phenol, and methyl salicylate. In some embodiments, the numbing agents include at least one of lidocaine, benzocaine, pramoxine, phenol, and methyl salicylate. In some embodiments, the hair restorative agents include at least one of minoxidil and finasteride. In some embodiments, the anti-itch agents include at least one of diphenhydramine, azelastine, olopatadine, ketotifen, and hydrocortisone. In some embodiments, the pungency agents include at least one of piperine, chavicine, isopiperine, dihydropiperine, and isochavicine. In some embodiments, the fluoride sources include at least one of sodium fluoride, stannous fluoride, and acidulated phosphate fluoride. In some embodiments, the one or more flavoring agents comprise one or more salts. In particular embodiments, the one or more salts comprise at least one of magnesium chloride, calcium chloride, and sodium bicarbonate. In some embodiments, the at least one non-gaseous solute comprises one or more electrolytes and minerals. In particular embodiments, the one or more electrolytes and minerals comprises magnesium, sodium, potassium, chloride, sulfate, benzoate, bicarbonate, zinc, or combinations thereof.

In some embodiments, the water is selected from DI water, ultrapure water, tap water, groundwater (e.g., well water), surface water, and reverse osmosis water. In particular embodiments, the water is ultrapure water. In particular embodiments, the water is DI water. In some embodiments, the water is tap water.

In some embodiments, the applied compositions include water having a population of ultrafine bubbles with a median ultrafine bubble diameter of between about 2-400 nanometers. In another embodiment, the ultrafine bubbles have a median diameter of between about 2 to about 10 nanometers (e.g., about 2 nanometers, about 3 nanometers, about 4 nanometers, about 5 nanometers, about 6 nanometers, about 7 nanometers, about 8 nanometers, about 9 nanometers, or about 10 nanometers). In other embodiments, the ultrafine bubbles have a median diameter of between about 10 to about 15 nanometers, about 15 to about 20 nanometers, or about 20 to about 25 nanometers. In other embodiments, the ultrafine bubbles have a median diameter of between about 10 to about 50 nanometers, about 20 to about 50 nanometers, about 30 to about 50 nanometers, or about 40 to about 50 nanometers. In still other embodiments, the ultrafine bubbles have a median diameter of between about 50 to about 100 nanometers. In yet further embodiments, the ultrafine bubbles have a median diameter of between about 100 to about 200 nanometers, about 150 to about 200 nanometers, about 200 to about 300 nanometers, about 250 to about 300 nanometers, or about 300 to about 400 nanometers.

In some embodiments, the ultrafine bubbles are present in the composition at a concentration of up to 10¹⁰ ultrafine bubbles/mL, as measured via nanoparticle tracking analysis (NTA), which is able to detect bubbles with diameters of 50 to 1000 nanometers. In some embodiments, the ultrafine bubbles are present in the composition at a range of 10 to 10² ultrafine bubbles/mL, 10² to 10³ ultrafine bubbles/mL, 10³ to 10⁴ ultrafine bubbles/mL, 10⁴ to 10⁵ ultrafine bubbles/mL, 10⁵ to 10⁶ ultrafine bubbles/mL, 10⁶ to 10⁷ ultrafine bubbles/mL, 10⁷ to 10⁸ ultrafine bubbles/mL, 10⁸ to 10⁹ ultrafine bubbles/mL, or 10⁹ to 10¹⁰ ultrafine bubbles/mL.

In certain embodiments in which the water of the composition is enriched via microbubble generation prior to generation of the ultrafine bubbles in the composition, the concentration of ultrafine bubbles in the resulting composition may be higher. In such embodiments, the ultrafine bubbles may be present in the composition at a range of 1010 to 1011 ultrafine bubbles/mL. In some embodiments, the ultrafine bubbles are present in the composition at a range of 10¹⁰ to 10¹¹ ultrafine bubbles/mL.

In some embodiments, the water has an oxidative reduction potential from about −200 mV to about 800 mV (e.g., about −200 mV, about −195 mV, about −190 mV, about −185 mV, about −180 mV, about −175 mV, about −170 mV, about −165 mV, about −160 mV, about −155 mV, about −150 mV, about −145 mV, about −140 mV, about −135 mV, about −130 mV, about −125 mV, about −120 mV, about −115 mV, about −110 mV, about −105 mV, about −100 mV, about −95 mV, about −90 mV, about −85 mV, about −80 mV, about −75 mV, about −70 mV, about −65 mV, about −60 mV, about −55 mV, about −50 mV, about −45 mV, about −40 mV, about −35 mV, about −30 mV, about −25 mV, about −20 mV, about −15 mV, about −10 mV, about −5 mV, about 0 mV, about 5 mV, about 10 mV, about 15 mV, about 20 mV, about 25 mV, about 30 mV, about 35 mV, about 40 mV, about 45 mV, about 50 mV, about 55 mV, about 60 mV, about 65 mV, about 70 mV, about 75 mV, about 80 mV, about 85 mV, about 90 mV, about 95 mV, about 100 mV, about 105 mV, about 110 mV, about 115 mV, about 120 mV, about 125 mV, about 130 mV, about 135 mV, about 140 mV, about 145 mV, about 150 mV, about 155 mV, about 160 mV, about 165 mV, about 170 mV, about 175 mV, about 180 mV, about 185 mV, about 190 mV, about 195 mV, about 200 mV, about 205 mV, about 210 mV, about 215 mV, about 220 mV, about 225 mV, about 230 mV, about 235 mV, about 240 mV, about 245 mV, about 250 mV, about 255 mV, about 260 mV, about 265 mV, about 275 mV, about 280 mV, about 290 mV, about 295 mV, about 300 mV, about 305 mV, about 310 mV, 315 mV, 320 mV, 325 mV, 330 mV, 335 mV, 340 mV, 345 mV, 350 mV, 355 mV, 360 mV, 365 mV, 370 mV, 375 mV, 380 mV, 385 mV, 390 mV, 395 mV, 400 mV, 405 mV, 410 mV, 415 mV, 420 mV, 425 mV, 430 mV, 435 mV, 440 mV, 445 mV, 450 mV, 455 mV, 460 mV, 465 mV, 470 mV, 475 mV, 480 mV, 485 mV, 490 mV, 495 mV, 500 mV, 505 mV, 510 mV, 515 mV, 520 mV, 525 mV, 530 mV, 535 mV, 540 mV, 545 mV, 550 mV, 555 mV, 560 mV, 565 mV, 570 mV, 575 mV, 580 mV, 585 mV, 590 mV, 595 mV, about 600 mV, about 605 mV, about 610 mV, about 615 mV, about 620 mV, about 625 mV, about 630 mV, about 635 mV, about 640 mV, about 645 mV, about 650 mV, about 655 mV, about 660 mV, about 665 mV, about 670 mV, about 675 mV, about 680 mV, about 685 mV, about 690 mV, about 695 mV, about 700 mV, about 705 mV, about 710 mV, about 715 mV, about 720 mV, about 725 mV, about 730 mV, about 735 mV, about 740 mV, about 745 mV, about 750 mV, about 755 mV, about 760 mV, about 765 mV, about 770 mV, about 775 mV, about 780 mV, about 785 mV, about 790 mV, about 795 mV, or about 800 mV).

In some embodiments, the pH of the water is between about 4 to about 8 (e.g., about 4, about 5, about 6, about 7, or about 8). In some embodiments of each or any of the above- or below-mentioned embodiments, the water has a resistivity between about 17 to about 18.2 meg-ohm cm.

In some embodiments, the compositions comprise ultrafine bubbles comprising or consisting essentially of water and gases released from solution in water, wherein the ultrafine bubbles optionally dissolve, surround, and/or stabilize a non-gaseous solute and/or a gaseous solute, and wherein the composition has a zeta potential of between about absolute value 0 and 40. In further embodiments, the zeta potential of the composition is between about −40 mV to about 0 mV. In still further embodiments, the zeta potential of the composition is between about −40 mV to about −35 mV, about −35 mV to about −30 mV, about −30 mV to about −25 mV, about −25 mV to about −20 mV, about −20 mV to about −15 mV, about −15 mV to about −10 mV, about −10 mV to about −5 mV, about −5 mV to about 0 mV, about 0 mV to about 5 mV, about 5 mV to about 10 mV, about 10 mV to about 15 mV, about 15 mV to about 20 mV, about 20 mV to about 25 mV, about 25 mV to about 30 mV, about 30 mV to about 35 mV, or about 35 mV to about 40 mV. The inventors have surprisingly found that despite relatively low zeta potentials of the compositions (e.g., typical ultrafine bubble compositions have zeta potentials of around absolute value 30 mV, significantly higher than the absolute value zeta potentials of the compositions herein), the ultrafine bubble compositions according to the disclosure herein achieve superior stability results over ultrafine bubbles formed by alternative means with higher absolute value zeta potentials.

In some embodiments, the applied compositions include at least one non-gaseous solute and/or at least one gaseous solute. In further embodiments, the at least one non-gaseous solute and/or the at least one gaseous solute is dissolved within, surrounded by, and/or stabilized by the ultrafine bubbles.

In some embodiments, the compositions comprise at least one non-gaseous solute and/or at least one gaseous solute (e.g., a solute dissolved within the composition). In further embodiments, the at least one non-gaseous solute and/or at least one gaseous solute is dissolved within, surrounded by, or stabilized by the ultrafine bubbles. In particular embodiments, the composition increases cell permeability and/or bioavailability of the at least one dissolved non-gaseous solute and/or at least one dissolved gaseous solute. In some embodiments, the at least one non-gaseous solute and/or at least one gaseous solute dissolved within or stabilized by the ultrafine bubbles has improved bioavailability relative to a solute not dissolved within or stabilized by the ultrafine bubbles. In further embodiments the at least one non-gaseous solute and/or at least one gaseous solute dissolved within or stabilized by the ultrafine bubbles has improved stability relative to a solute not dissolved within or stabilized by the ultrafine bubbles. In still further embodiments, the at least one non-gaseous solute and/or at least one gaseous solute dissolved within or stabilized by the ultrafine bubbles has improved solubility relative to a solute not dissolved within or stabilized by the ultrafine bubbles.

In some embodiments, the composition is used to deliver the at least one non-gaseous solute and/or at least one gaseous solute to a cell (e.g., the interior of the cell). In some embodiments, the cell is an animal cell. In some embodiments, the cell is a mammalian cell. In some embodiments, the cell is a human cell. In some embodiments, the cell is a skin or epithelial cell.

In some embodiments, the ultrafine bubbles (e.g., ultrafine bubbles comprising gases released from solution in water) are concentrated within the composition via rotary evaporation and/or cross flow filtration.

In some embodiments, the compositions and/or ultrafine bubbles are stable and/or exhibit biological efficacy for at least six months, for at least one year, for at least 2 years, for at least 3 years, for at least 4 years, or for at least 5 years.

In some embodiments of each or any of the above- or below-mentioned embodiments, the composition is used for a consumer products application. The present disclosure also provides a composition that includes ultrafine bubbles comprising or consisting essentially of water and gases released from solution in the water, wherein the ultrafine bubbles have a median ultrafine bubble diameter of between about 2 to about 400 nanometers, and wherein the ultrafine bubbles dissolve, surround, and/or stabilize a non-gaseous solute and/or a gaseous solute. In another embodiment, the ultrafine bubbles have a median size of between about 2 to about 10 nanometers (e.g., about 2 nanometers, about 3 nanometers, about 4 nanometers, about 5 nanometers, about 6 nanometers, about 7 nanometers, about 8 nanometers, about 9 nanometers, or about 10 nanometers). In other embodiments, the ultrafine bubbles have a median size of between about 10 to about 20 nanometers or about 15 to about 20 nanometers, or about 20 to about 25 nanometers. In other embodiments, the ultrafine bubbles have a median size of between about 10 to about 50 nanometers, about 20 to about 50 nanometers, about 30 to about 50 nanometers, or about 40 to about 50 nanometers. In still other embodiments, the ultrafine bubbles have a median size of between about 50 to about 100 nanometers. In yet further embodiments, the ultrafine bubbles have a median size of between about 100 to about 200 nanometers, about 150 to about 200 nanometers, about 200 to about 300 nanometers, about 250 to about 300 nanometers, or about 300 to about 400 nanometers.

In some embodiments of each or any of the above- or below-mentioned embodiments, the composition is used to deliver at least one non-gaseous solute and/or at least one gaseous solute to the interior of a cell (e.g., an animal cell). In some embodiments, the composition includes ultrafine bubbles comprising or consisting essentially of water and gases released from solution in the water, wherein the ultrafine bubbles dissolve, surround, and/or stabilize the at least one non-gaseous solute and/or at least one gaseous solute (e.g., flavoring agents).

In another aspect of the invention disclosed herein, a method for producing a composition comprising water and ultrafine bubbles including gases released from solution in the water is provided. In some embodiments, the ultrafine bubbles are at a concentration of up to 1010 ultrafine bubbles/mL. In other embodiments, the ultrafine bubbles have a concentration of up to 1011 ultrafine bubbles/mL. The method includes subjecting water to a combination of hydrodynamic cavitation, shear forces, and thin film boiling to produce ultrafine bubbles formed by release of dissolved gases from the water. In some embodiments, the water is selected from DI water, ultrapure water, tap water, groundwater (e.g., well water), surface water, and reverse osmosis water. In particular embodiments, the water is ultrapure water. In other embodiments, the water is tap water. In some embodiments, the ultrafine bubbles are present in the composition at a range of 10 to 10² ultrafine bubbles/mL, 10² to 10³ ultrafine bubbles/mL, 10³ to 10⁴ ultrafine bubbles/mL, 10⁴ to 10⁵ ultrafine bubbles/mL, 10⁵ to 10⁶ ultrafine bubbles/mL, 10⁶ to 10⁷ ultrafine bubbles/mL, 10⁷ to 10⁸ ultrafine bubbles/mL, 10⁸ to 10⁹ ultrafine bubbles/mL, 10⁹ to 10¹⁰ ultrafine bubbles/mL, or 10¹⁰ to 10¹¹ ultrafine bubbles/mL.

In an embodiment of the method, the method further comprises dissolving at least one non-gaseous solute and/or at one least gaseous solute into the composition. In some embodiments, the at least one non-gaseous solute and/or at least gaseous solute is dissolved within, surrounded by, and/or stabilized by the ultrafine bubbles.

In some embodiments, the methods further comprise concentrating the ultrafine bubbles within the composition via rotary evaporation or cross flow filtration.

In some embodiments, the ultrafine bubbles comprise about 25, about 30, about 35, about 40, about 45, about 50, about 75, about 100, about 125, about 150, about 175, about 200, about 225, about 250, about 275, about 300, about 325, about 350, about 375, about 400, about 425, about 450, about 475, or about 500 water molecules.

In some embodiments, the ultrafine bubbles have a median size (diameter) of between about 2 to about 400 nanometers. In another embodiment, the ultrafine bubbles have a median size of between about 2 to about 10 nanometers (e.g., about 2 nanometers, about 3 nanometers, about 4 nanometers, about 5 nanometers, about 6 nanometers, about 7 nanometers, about 8 nanometers, about 9 nanometers, or about 10 nanometers). In other embodiments, the ultrafine bubbles have a median size of between about 10 to about 15 nanometers or about 15 to about 20 nanometers, or about 20 to about 25 nanometers. In other embodiments, the ultrafine bubbles have a median size of between about 10 to about 50 nanometers, about 20 to about 50 nanometers, about 30 to about 50 nanometers, or about 40 to about 50 nanometers. In still other embodiments, the ultrafine bubbles have a median size of between about 50 to about 100 nanometers. In yet further embodiments, the ultrafine bubbles have a median size of between about 100 to about 200 nanometers, about 150 to about 200 nanometers, about 200 to about 300 nanometers, about 250 to about 300 nanometers, or about 300 to about 400 nanometers.

In certain embodiments, the methods further include preparing the compositions used by pumping the water and the one or more non-gaseous solutes and/or at least one or more gaseous solutes through a transfer pipe and a nozzle into a hollow cylinder, wherein the nozzle is located at the proximal end of the hollow cylinder. The nozzle includes an intake hole in a proximal face of the nozzle connected to the transfer pipe and one or more jet openings in a distal face of the nozzle that open into a chamber defined by the hollow cylinder. The water passing through the one or more jet openings creates a vortex of water in contact with an inner surface of the chamber. The compositions exit the hollow cylinder (containing ultrafine bubbles and the one or more non-gaseous solutes as disclosed), such that the one or more non-gaseous solutes and/or at least one or more gaseous solutes is dissolved within, surrounded by, and/or stabilized by the ultrafine bubbles in accordance with the disclosure. Further non-gaseous solutes and/or gaseous solutes may be added to the compositions after exiting the hollow cylinder.

In other embodiments, the methods further include preparing the compositions used by pumping the water through a transfer pipe and a nozzle into a hollow cylinder, wherein the nozzle is located at the proximal end of the hollow cylinder. The nozzle includes an intake hole in a proximal face of the nozzle connected to the transfer pipe and one or more jet openings in a distal face of the nozzle that open into a chamber defined by the hollow cylinder. The water passing through the one or more jet openings creates a vortex of water in contact with an inner surface of the chamber. The pumped water exits the hollow cylinder (containing ultrafine bubbles as disclosed), after which the one or more non-gaseous solutes and/or one or more gaseous solutes is optionally mixed into the exited water to produce a composition in which the one or more non-gaseous solutes and/or one or more gaseous solutes is dissolved within, surrounded by, and/or stabilized by ultrafine bubbles in accordance with this disclosure.

In an embodiment, the composition or solution is stable for at least about 2 years. In some embodiments, the ultrafine bubbles are stable for about 2 years, about 2.5 years, about 3 years, about 4 years, about 5 years, about 6 years, about 7 years, about 8 years, about 9 years, or about 10 years. In some embodiments, the ultrafine bubbles are stable for a period in excess of 10 years.

In some embodiments of each or any of the above- or below-mentioned embodiments, the ultrafine bubbles comprise or consist essentially of ultrapure water having an oxidative reduction potential about −200 to about 800 mV (e.g., from about −200 mV to about 800 mV (e.g., about −200 mV, about −195 mV, about −190 mV, about −185 mV, about −180 mV, about −175 mV, about −170 mV, about −165 mV, about −160 mV, about −155 mV, about −150 mV, about −145 mV, about −140 mV, about −135 mV, about −130 mV, about −125 mV, about −120 mV, about −115 mV, about −110 mV, about −105 mV, about −100 mV, about −95 mV, about −90 mV, about −85 mV, about −80 mV, about −75 mV, about −70 mV, about −65 mV, about −60 mV, about −55 mV, about −50 mV, about −45 mV, about −40 mV, about −35 mV, about −30 mV, about −25 mV, about −20 mV, about −15 mV, about −10 mV, about −5 mV, about 0 mV, about 5 mV, about 10 mV, about 15 mV, about 20 mV, about 25 mV, about 30 mV, about 35 mV, about 40 mV, about 45 mV, about 50 mV, about 55 mV, about 60 mV, about 65 mV, about 70 mV, about 75 mV, about 80 mV, about 85 mV, about 90 mV, about 95 mV, about 100 mV, about 105 mV, about 110 mV, about 115 mV, about 120 mV, about 125 mV, about 130 mV, about 135 mV, about 140 mV, about 145 mV, about 150 mV, about 155 mV, about 160 mV, about 165 mV, about 170 mV, about 175 mV, about 180 mV, about 185 mV, about 190 mV, about 195 mV, or about 200 mV, about 205 mV, about 210 mV, about 215 mV, about 220 mV, about 225 mV, about 230 mV, about 235 mV, about 240 mV, about 245 mV, about 250 mV, about 255 mV, about 260 mV, about 265 mV, about 275 mV, about 280 mV, about 290 mV, about 295 mV, about 300 mV, about 305 mV, about 310 mV, 315 mV, 320 mV, 325 mV, 330 mV, 335 mV, 340 mV, 345 mV, 350 mV, 355 mV, 360 mV, 365 mV, 370 mV, 375 mV, 380 mV, 385 mV, 390 mV, 395 mV, 400 mV, 405 mV, 410 mV, 415 mV, 420 mV, 425 mV, 430 mV, 435 mV, 440 mV, 445 mV, 450 mV, 455 mV, 460 mV, 465 mV, 470 mV, 475 mV, 480 mV, 485 mV, 490 mV, 495 mV, 500 mV, 505 mV, 510 mV, 515 mV, 520 mV, 525 mV, 530 mV, 535 mV, 540 mV, 545 mV, 550 mV, 555 mV, 560 mV, 565 mV, 570 mV, 575 mV, 580 mV, 585 mV, 590 mV, 595 mV, about 600 mV, about 605 mV, about 610 mV, about 615 mV, about 620 mV, about 625 mV, about 630 mV, about 635 mV, about 640 mV, about 645 mV, about 650 mV, about 655 mV, about 660 mV, about 665 mV, about 670 mV, about 675 mV, about 680 mV, about 685 mV, about 690 mV, about 695 mV, about 700 mV, about 705 mV, about 710 mV, about 715 mV, about 720 mV, about 725 mV, about 730 mV, about 735 mV, about 740 mV, about 745 mV, about 750 mV, about 755 mV, about 760 mV, about 765 mV, about 770 mV, about 775 mV, about 780 mV, about 785 mV, about 790 mV, about 795 mV, or about 800 mV).

In still further embodiments, the pH of the water is between about 4 to about 8 (e.g., about 4, about 5, about 6, about 7, or about 8). In some embodiments of each or any of the above- or below-mentioned embodiments, the composition or solution is used in the method to deliver a nutrient solute to the interior of a cell (e.g., a plant cell).

The present disclosure also provides a method of using a composition or solution that includes ultrafine bubbles that comprise or consist essentially of water molecules surrounding the gases released from solution in the water dissolving, surrounding, and/or stabilizing a solute (e.g., a dietary nutrient), wherein the ultrafine bubbles have a median diameter of between about 2 to about 400 nanometers, and wherein the composition including such ultrafine bubbles has improved bioavailability relative to a composition or a solution that does not include ultrafine bubbles that comprise or consist essentially of water molecules surrounding the gases released from solution in the water. In some embodiments, the ultrafine bubbles have a median of about 150 to about 300 water molecules per ultrafine bubble. In other embodiments, the ultrafine bubbles have a median of about 25, about 30, about 35, about 40, about 45, about 50, about 75, about 100, about 125, about 150, about 175, about 200, about 225, about 250, about 275, about 300, about 325, about 350, about 375, about 400, about 425, about 450, about 475, or about 500 water molecules per ultrafine bubble.

The present disclosure also provides methods for improving the bioavailability of a solute (e.g., a dietary nutrient). In certain embodiments, the methods comprise dissolving the solute in water and dissolving/surrounding/stabilizing the solute with ultrafine bubbles, wherein the ultrafine bubbles are between about 2 to about 400 nanometers in median diameter. In another embodiment, the ultrafine bubbles have a median size of between about 2 to about 10 nanometers (e.g., about 2 nanometers, about 3 nanometers, about 4 nanometers, about 5 nanometers, about 6 nanometers, about 7 nanometers, about 8 nanometers, about 9 nanometers, or about 10 nanometers). In other embodiments, the ultrafine bubbles have a median size of between about 10 to about 20 nanometers or about 15 to about 20 nanometers, or about 20 to about 25 nanometers. In other embodiments, the ultrafine bubbles have a median size of between about 10 to about 50 nanometers, about 20 to about 50 nanometers, about 30 to about 50 nanometers, or about 40 to about 50 nanometers. In still other embodiments, the ultrafine bubbles have a median size of between about 50 to about 100 nanometers. In yet further embodiments, the ultrafine bubbles have a median size of between about 100 to about 200 nanometers, about 150 to about 200 nanometers, about 200 to about 300 nanometers, about 250 to about 300 nanometers, or about 300 to about 400 nanometers.

The present disclosure also provides methods for dissolving, surrounding, and/or stabilizing a solute in water comprising mixing the solute with water and dissolving/surrounding/stabilizing the solute with ultrafine bubbles having a median diameter of between about 2 to about 400 nanometers. In another embodiment, the ultrafine bubbles have a median size of between about 2 to about 10 nanometers (e.g., about 2 nanometers, about 3 nanometers, about 4 nanometers, about 5 nanometers, about 6 nanometers, about 7 nanometers, about 8 nanometers, about 9 nanometers, or about 10 nanometers). In other embodiments, the ultrafine bubbles have a median size of between about 10 to about 20 nanometers or about 15 to about 20 nanometers, or about 20 to about 25 nanometers. In other embodiments, the ultrafine bubbles have a median size of between about 10 to about 50 nanometers, about 20 to about 50 nanometers, about 30 to about 50 nanometers, or about 40 to about 50 nanometers. In still other embodiments, the ultrafine bubbles have a median size of between about 50 to about 100 nanometers. In yet further embodiments, the ultrafine bubbles have a median size of between about 100 to about 200 nanometers, about 150 to about 200 nanometers, about 200 to about 300 nanometers, about 250 to about 300 nanometers, or about 300 to about 400 nanometers.

### BRIEF DESCRIPTION OF THE FIGURES

FIG. 1 shows a diagram of a system (101) and a method for making compositions including water and ultrafine bubbles in accordance with embodiments of the disclosure.

FIG. 2 shows an overlaid Raman finger-print region spectra of niacinamide and human skin in accordance with embodiments of the disclosure.

FIG. 3 shows a Raman hyperspectral image comparing control skin samples, skin samples treated with Formulation 1, and skin samples treated with Formulation 2 for niacinamide content in accordance with embodiments of the disclosure (measurements taken 2 months post-treatment).

FIG. 4 shows a Raman hyperspectral image comparing control skin samples, skin samples treated with Formulation 1, and skin samples treated with Formulation 2 for niacinamide content in accordance with embodiments of the disclosure (measurements taken 2 months post-treatment).

FIG. 5 shows a Raman hyperspectral image comparing control skin samples, skin samples treated with Formulation 1, and skin samples treated with Formulation 2 for niacinamide content in accordance with embodiments of the disclosure (measurements taken 18 months post-treatment).

FIG. 6 shows an overlaid Raman finger-print region spectra of bakuchiol and human skin in accordance with embodiments of the disclosure.

FIG. 7 shows a Raman hyperspectral image comparing control skin samples, skin samples treated with Formulation 3, and skin samples treated with Formulation 4 for bakuchiol content in accordance with embodiments of the disclosure (measurements taken 2 months post-treatment).

FIG. 8 shows an overlaid Raman finger-print region spectra of Everwhite active and human skin in accordance with embodiments of the disclosure.

FIG. 9 shows a Raman hyperspectral image comparing control skin samples, skin samples treated with Formulation 5, and skin samples treated with Formulation 6 for Everwhite active content in accordance with embodiments of the disclosure (measurements taken 2 months post-treatment).

FIG. 10 shows a Raman hyperspectral image comparing control skin samples, skin samples treated with Formulation 1, and skin samples treated with Formulation 2 (i.e., the niacinamide samples) for water content in accordance with embodiments of the disclosure (measurements taken 2 months post-treatment).

FIG. 11 shows a Raman hyperspectral image comparing control skin samples, skin samples treated with Formulation 1, and skin samples treated with Formulation 2 (i.e., the niacinamide samples) for water content in accordance with embodiments of the disclosure (measurements taken 18 months post-treatment).

FIG. 12 shows a Raman hyperspectral image comparing control skin samples, skin samples treated with Formulation 3, and skin samples treated with Formulation 4 (i.e., the bakuchiol samples) for water content in accordance with embodiments of the disclosure (measurements taken 2 months post-treatment).

FIG. 13 shows a Raman hyperspectral image comparing control skin samples, skin samples treated with Formulation 5, and skin samples treated with Formulation 6 (i.e., the Everwhite samples) for water content in accordance with embodiments of the disclosure (measurements taken 2 months post-treatment).

FIG. 14 depicts a graph of total water content for each of Formulations 1-6 in comparison to Control in the skin samples in accordance with embodiments of the disclosure (measurements taken 2 months post-treatment).

FIG. 15 depicts a graph of water content for each of Formulations 1-6 in comparison to Control in the skin samples, wherein the area above the stratum corneum has been “masked” from the calculation of water content to exclude the film-formation of water outside of the stratum corneum, in accordance with embodiments of the disclosure (measurements taken 2 months post-treatment).

FIG. 16 depicts a graph of total water content for Formulations 1 and 2 (measurements were duplicated for both Formulations) in comparison to Control in the skin samples in accordance with embodiments of the disclosure (measurements taken 18 months post-treatment).

FIG. 17 depicts a graph of niacinamide distribution in a control formulation (“control”), a DI Water formulation (DI), and an ultrafine bubble suspension test formulation (UFB), based on the 1035 cm-1 band height relative to the 1650 cm-1 band in accordance with embodiments of the disclosure.

### DETAILED DESCRIPTION

The present disclosure provides compositions and methods for using the compositions and solutions (e.g., aqueous compositions) that include ultrafine bubbles comprising or consisting essentially of water and gases released from solution in the water, and optionally one or more non-gaseous solutes and/or one or more gaseous solutes. The inventors have surprisingly found that compositions according to the disclosure herein can be applied to consumer applications (e.g., beverages, oral care solutions, food compositions, skin care products). Indeed, the inventors have surprisingly discovered that aqueous compositions that comprise a low concentration of ultrafine bubbles (e.g., at a concentration of up to 10⁸ ultrafine bubbles/mL) exert improved/increased bioavailability, solubility, permeability with respect to biological membranes, and/or stability than previously anticipated, perhaps even as compared to compositions that comprise a higher concentration of ultrafine bubbles (e.g., more than 10⁸ ultrafine bubbles/mL).

The ultrafine bubbles may comprise or consist essentially of water and gases released from solution in the water. The ultrafine bubbles may be used advantageously to dissolve, surround, and/or stabilize a non-gaseous solute (e.g., a dietary nutrient, an organic chemical, an inorganic chemical, or a flavoring agent) and used to deliver the solute across cellular membranes or barriers of a cell (e.g., a skin cell) to exert its effect. As such, the disclosed compositions and solutions provide surprising and unexpected advantages in promoting absorption and/or uptake of solutes or water based, for example, on the improved bioavailability, solubility, and/or stability of ultrafine bubbles comprising or consisting essentially of water and gases released from solution in the water, and the optional non-gaseous solutes included within the composition including the ultrafine bubbles.

Also provided by the present disclosure are methods for making aqueous ultrafine bubbles (e.g., from ultrapure water), including methods for dissolving a solute (e.g., a dietary nutrient) in an aqueous composition including ultrafine bubbles comprising or consisting essentially of water and gases released from solution in the water. The ultrafine bubbles comprising or consisting essentially of water and gases released from solution in the water may be used to dissolve, stabilize, and/or surround solutes (e.g., dietary nutrients, inorganic chemicals, flavoring agents, mineral, organic chemicals). In certain embodiments of the methods, the ultrafine bubbles in the composition have a median size of between about 2 to about 400 nanometers. In another embodiment, the ultrafine bubbles have a median size of between about 2 to about 10 nanometers (e.g., about 2 nanometers, about 3 nanometers, about 4 nanometers, about 5 nanometers, about 6 nanometers, about 7 nanometers, about 8 nanometers, about 9 nanometers, or about 10 nanometers). In other embodiments, the ultrafine bubbles have a median size of between about 10 to about 20 nanometers or about 15 to about 20 nanometers, or about 20 to about 25 nanometers. In other embodiments, the ultrafine bubbles have a median size of between about 10 to about 50 nanometers, about 20 to about 50 nanometers, about 30 to about 50 nanometers, or about 40 to about 50 nanometers. In still other embodiments, the ultrafine bubbles have a median size of between about 50 to about 100 nanometers. In yet further embodiments, the ultrafine bubbles have a median size of between about 100 to about 200 nanometers, about 150 to about 200 nanometers, about 200 to about 300 nanometers, about 250 to about 300 nanometers, or about 300 to about 400 nanometers.

The present disclosure also provides compositions and solutions used in the methods wherein 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the composition or solution includes ultrafine bubbles comprising or consisting essentially of water and gases released from solution in the water and a solute, wherein the ultrafine bubbles dissolve, surround, and/or stabilize the solute.

In some embodiments of the methods, about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ultrafine bubbles comprising or consisting essentially of water and gases released from solution in the water in the composition or solution dissolve, surround, and/or stabilize the solute.

In some embodiments of the methods, one or more of the solutes is present at a concentration of from about 1 mg/L to about 1000 mg/L of composition according to the disclosure herein. Compositions and solutions used in the methods herein include the one or more solutes at a concentration of about 1 mg/L, about 2 mg/L, about 3 mg/L, about 4 mg/L, about 5 mg/L, about 10 mg/L, about 20 mg/L, about 30 mg/L, about 40 mg/L, about 50 mg/L, about 60 mg/L, about 70 mg/L, about 80 mg/L, about 90 mg/L, about 100 mg/L, about 110 mg/L, about 120 mg/L, about 130 mg/L, about 140 mg/L, about 150 mg/L, about 160 mg/L, about 170 mg/L, about 180 mg/L, about 190 mg/L, about 200 mg/L, about 210 mg/L, about 220 mg/L, about 230 mg/L, about 240 mg/L, about 250 mg/L, about 260 mg/L, about 270 mg/L, about 280 mg/L, about 290 mg/L, about 300 mg/L, about 310 mg/L, about 320 mg/L, about 330 mg/L, about 340 mg/L, about 350 mg/L, about 360 mg/L, about 370 mg/L, about 380 mg/L, about 390 mg/L, about 400 mg/L, about 410 mg/L, about 420 mg/L, about 430 mg/L, about 440 mg/L, about 450 mg/L, about 460 mg/L, about 470 mg/L, about 480 mg/L, about 490 mg/L, about 500 mg/L, about 510 mg/L, about 520 mg/L, about 530 mg/L, about 540 mg/L, about 550 mg/L, about 560 mg/L, about 570 mg/L, about 580 mg/L, about 590 mg/L, about 600 mg/L, about 610 mg/L, about 620 mg/L, about 630 mg/L, about 640 mg/L, about 650 mg/L, about 660 mg/L, about 670 mg/L, about 680 mg/L, about 690 mg/L, about 700 mg/L, about 710 mg/L, about 720 mg/L, about 730 mg/L, about 740 mg/L, about 750 mg/L, about 760 mg/L, about 770 mg/L, about 780 mg/L, about 790 mg/L, about 800 mg/L, about 810 mg/L, about 820 mg/L, about 830 mg/L, about 840 mg/L, about 850 mg/L, about 860 mg/L, about 870 mg/L, about 880 mg/L, about 890 mg/L, about 900 mg/L, about 910 mg/L, about 920 mg/L, about 930 mg/L, about 940 mg/L, about 950 mg/L, about 960 mg/L, about 970 mg/L, about 980 mg/L, about 990 mg/L, or about 1000 mg/L.

Without being bound by theory, it is believed the compositions as disclosed herein provide for improved bioavailability, solubility, and/or stability of the ultrafine bubbles comprising or consisting essentially of water and gases released from solution in water, as well as improved bioavailability, solubility, and/or stability of any dissolved solutes because the ultrafine bubbles are produced from “soft” or gaseous cavitation rather than “hard” or vaporous cavitation processes. The disclosed ultrafine bubbles are believed to be (a) nucleated in the low-pressure vicinity surrounding the cavitation core, (b) sheared-off bubbles from the cavitation core itself, or (c) produced via low pressure/room temperature boiling at the core surface, such that, in the presence of turbulence and high shear stresses near the core, ultrafine bubbles are broken into smaller ultrafine bubbles through deformation (due to drag forces). The resulting compositions incorporating such ultrafine bubbles exhibit improved efficacy for dissolving solutes, even at concentrations of 107 ultrafine bubbles/mL and below. Such compositions also exhibit enhanced stability over other solutions incorporating ultrafine bubbles or ultrafine bubbles produced via other means, as they can be concentrated by several orders of magnitude via rotary evaporation or crossflow filtration without ultrafine bubble loss or solute dissolution, and can even remain bottled for up to 10 years without loss of ultrafine bubble concentration or dissolution of solutes.

Where the term “comprising” is used in the present description and the claims, it does not exclude other elements or steps. For the purposes of the present invention, the term “consisting of” is considered to be a preferred embodiment of the term “comprising”.

Specific embodiments disclosed herein can be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

In cases where numerical values are indicated in the context of the present disclosure, the skilled person will understand that the technical effect of the feature in question is ensured within an interval of accuracy, which typically encompasses a deviation of the numerical value given of ±10%, and preferably of ±5%. At the very least, and not as an attempt to limit the application of the doctrine of equivalents to the scope of the claims, each numerical parameter should at least be construed in light of the number of reported significant digits and by applying ordinary rounding techniques.

Unless otherwise indicated, all numbers expressing quantities of ingredients, properties such as molecular weight and median size, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified, thus fulfilling the written description of all Markush groups used in the appended claims.

Certain embodiments of this disclosure are described herein, including the best mode known to the inventor for carrying out the disclosure. Of course, variations on these described embodiments will become apparent to those of ordinary skill in the art upon reading the foregoing description. The inventor expects skilled artisans to employ such variations as appropriate, and the inventor intends for the disclosure to be practiced other than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entirety.

Further definitions of terms will be given in the following in the context of which the terms are used. The following terms or definitions are provided solely to aid in the understanding of the invention. These definitions should not be construed to have a scope less than understood by a person of ordinary skill in the art.

As used herein, an “ultrafine bubble” refers to an assembly of water molecules, with a diameter less than one micron, bonded with or otherwise associated with one another by electrostatic forces, such as hydrogen bonding, ionic bonding, van der Waals forces, or the like, surrounding gases (e.g., gases released from solution in water). In some cases according to the disclosure, an ultrafine bubble further comprises a non-gaseous solute associated with the water molecules and dissolved within, surrounded by, and/or stabilized by the ultrafine bubble.

As used herein, a “solute” means a substance or particle that is fully or partially dissolved in water. In embodiments, a solute of the disclosure is dissolved within, surrounded by, and/or stabilized by ultrafine bubbles of the disclosure. A solute according to the disclosure comprises, without limitation, a plant nutrient, an ion, a polar or non-polar substance, a liquid, a solid, a lipid, a protein, a peptide, a nucleic acid, an organic compound, an inorganic compound, a gas, or any combination thereof.

As used herein, “ultrapure water” means water prepared according to one or more of the described embodiments of the disclosure. In particular, ultrapure water refers to water prepared by methods and processes disclosed herein, or water characterized as being completely free of (e.g., does not contain any detectable amount), or substantially free of (e.g., 70%, 80%, 90%, or 95% free of), one or more impurities or contaminants.

As used herein, “bioavailability” refers to the physiological availability of a given amount of a solute as distinct from its chemical potency. For example, bioavailability refers to the proportion of an administered solute that is absorbed into the tissues of a plant (e.g., a cannabis plant). Bioavailability also refers to the ability of an ultrafine bubble, solute, particle, dissolved solute, or combination thereof, to access a biological target, e.g., by crossing a biological membrane or by interacting with a biological receptor or other binding partner.

The disclosure provides methods using compositions and solutions comprising ultrafine bubbles comprising or consisting essentially of water and gases released from solution in the water, and a non-gaseous solute dissolved within, surrounded by, and/or stabilized by the ultrafine bubbles. The ultrafine bubble may have a median ultrafine bubble size of between about 2 to about 400 nanometers or a median of about 10 to about 500 water molecules per ultrafine bubble.

In one aspect of the invention, the compositions are aqueous compositions for oral administration (e.g., mouthwashes) and/or ingestion (e.g., beverages). The compositions include water and ultrafine bubbles comprising gases released from solution in the water. In some embodiments, the composition increases cell permeability and/or bioavailability of the water within the composition. In some embodiments, the compositions include one or more non-gaseous solutes (e.g., flavoring agents, antibacterial agents, fluoride sources, nutrients, electrolytes, minerals, warming-sensation agents, and cooling-sensation agents).

In some embodiments, the water is selected from DI water, ultrapure water, tap water, groundwater, surface water, and reverse osmosis water. In some embodiments, the water has a resistivity between about 17 to about 18.2 meg-ohm cm. In further embodiments, the water has a pH of between about 3 to about 7. In some embodiments, the water has an oxidative reduction potential of about −200 mV to about 800 mV.

In some embodiments, the aqueous compositions including ultrafine bubbles comprising or consisting essentially of water and gases released from solution in the water have improved bioavailability relative to naturally occurring water, and relative to compositions including ultrafine bubbles not formed via gaseous cavitation. In some embodiments, the ultrafine bubbles comprising or consisting essentially of water and gases released from solution in the water improve bioavailability of the water by about 10%, about 20%, about 30%, about 40%, about 50%, about 60%, about 70%, about 80%, or about 90% relative to naturally occurring water, and/or relative to compositions including ultrafine bubbles not formed via gaseous cavitation. In further embodiments, the ultrafine bubbles comprising or consisting essentially of water and dissolved/surrounded/stabilized solutes improve bioavailability of the water by about 1%, about 2%, about 3%, about 4%, about 5%, about 6%, about 7%, about 8%, or about 9% relative to naturally occurring water, and/or relative to compositions including ultrafine bubbles not formed via gaseous cavitation. In some embodiments, the at least one non-gaseous solute is dissolved within, surrounded by, and/or stabilized by the ultrafine bubbles. In some embodiments, the composition increases cell permeability and/or bioavailability of the at least one dissolved non-gaseous solute. In some embodiments, the at least one non-gaseous solute is present at a concentration of 0.1-30% by weight of the composition. In other embodiments, the at least one non-gaseous solute is present at a concentration of 0.1-1% by weight of the composition, 1-2% by weight of the composition, 2-3% by weight of the composition, 3-4% by weight of the composition, 4-5% by weight of the composition, 5-6% by weight of the composition, 6-7% by weight of the composition, 7-8% by weight of the composition, 8-9% by weight of the composition, 9-10% by weight of the composition, 10-11% by weight of the composition, 11-12% by weight of the composition, 12-13% by weight of the composition, 13-14% by weight of the composition, 14-15% by weight of the composition, 15-16% by weight of the composition, 16-17% by weight of the composition, 17-18% by weight of the composition, 18-19% by weight of the composition, 19-20% by weight of the composition, 20-21% by weight of the composition, 21-22% by weight of the composition, 22-23% by weight of the composition, 23-24% by weight of the composition, 24-25% by weight of the composition, 25-26% by weight of the composition, 26-27% by weight of the composition, 27-28% by weight of the composition, 28-29% by weight of the composition, or 29-30% by weight of the composition.

In some embodiments, the at least one non-gaseous solute and/or at least one gaseous solute is present at a low concentration of 0.1-10% by weight of the composition. In other embodiments, the at least one non-gaseous solute and/or at least one gaseous solute is present at a concentration of 0.1-0.5% by weight of the composition, 0.5-1% by weight of the composition, 1-1.5% by weight of the composition, 1.5-2% by weight of the composition, 2-2.5% by weight of the composition, 2.5-3% by weight of the composition, 3-3.5% by weight of the composition, 3.5-4% by weight of the composition, 4-4.5% by weight of the composition, 4.5-5% by weight of the composition, 5-5.5% by weight of the composition, 5.5-6% by weight of the composition, 6-6.5% by weight of the composition, 6.5-7% by weight of the composition, 7-7.5% by weight of the composition, 7.5-8% by weight of the composition, 8-8.5% by weight of the composition, 8.5-9% by weight of the composition, 9-9.5% by weight of the composition, or 9.5-10% by weight of the composition.

In some embodiments, the at least one non-gaseous solute and/or at least one gaseous solute is present at a medium concentration of 10-30% by weight of the composition. In other embodiments, the at least one non-gaseous solute and/or at least one gaseous solute is present at a concentration of 10-10.5% by weight of the composition, 10.5-11% by weight of the composition, 11-11.5% by weight of the composition, 11.5-12% by weight of the composition, 12-12.5% by weight of the composition, 12.5-13% by weight of the composition, 13-13.5% by weight of the composition, 13.5-14% by weight of the composition, 14-14.5% by weight of the composition, 14.5-15% by weight of the composition, 15-15.5% by weight of the composition, 15.5-16% by weight of the composition, 16-16.5% by weight of the composition, 16.5-17% by weight of the composition, 17-17.5% by weight of the composition, 17.5-18% by weight of the composition, 18-18.5% by weight of the composition, 18.5-19% by weight of the composition, 19-19.5% by weight of the composition, 19.5-20% by weight of the composition, 20-20.5% by weight of the composition, 20.5-21% by weight of the composition, 21-21.5% by weight of the composition, 21.5-22% by weight of the composition, 22-22.5% by weight of the composition, 22.5-23% by weight of the composition, 23-23.5% by weight of the composition, 23.5-24% by weight of the composition, 24-24.5% by weight of the composition, 24.5-25% by weight of the composition, 25-25.5% by weight of the composition, 25.5-26% by weight of the composition, 26-26.5% by weight of the composition, 26.5-27% by weight of the composition, 27-27.5% by weight of the composition, 27.5-28% by weight of the composition, 28-28.5% by weight of the composition, 28.5-29% by weight of the composition, 29-29.5% by weight of the composition, or 29.5-30% by weight of the composition.

In some embodiments, the at least one non-gaseous solute and/or at least one gaseous solute is present at a high concentration of 30-95% by weight of the composition. In other embodiments, the at least one non-gaseous solute and/or at least one gaseous solute is present at a concentration of 30-35% by weight of the composition, 35-40% by weight of the composition, 40-45% by weight of the composition, 45-50% by weight of the composition, 50-55% by weight of the composition, 55-60% by weight of the composition, 60-65% by weight of the composition, 65-70% by weight of the composition, 70-75% by weight of the composition, 75-80% by weight of the composition, 80-85% by weight of the composition, 85-90% by weight of the composition, or 90-95% by weight of the composition.

In some embodiments, the at least one non-gaseous solute and/or at least one gaseous solute is stable within the composition for at least 2 years.

In some embodiments, the composition comprising or consisting essentially of a non-gaseous solute and/or a gaseous solute, water, and gases released from solution in the water, wherein the ultrafine bubbles dissolve/surround/stabilize the non-gaseous solute and/or the gaseous solute, improves bioavailability of the solute by about 10%, about 20%, about 30%, about 40%, about 50%, about 60%, about 70%, about 80%, or about 90% relative to the undissolved/unsurrounded/unstabilized solute. In further embodiments, the ultrafine bubbles comprising or consisting essentially of water and non-gaseous solute(s) and/or gaseous solute(s) improve bioavailability of the solutes by about 1%, about 2%, about 3%, about 4%, about 5%, about 6%, about 7%, about 8%, or about 9%.

In some embodiments, the ultrafine bubbles have a median diameter of between 2-400 nanometers. In some embodiments, the ultrafine bubbles remain stable within the composition for at least two years. In particular embodiments, the ultrafine bubbles remain stable within the composition for at least 2.5 years. In some embodiments, the ultrafine bubbles are concentrated within the composition via rotary evaporation and/or cross flow filtration. In particular embodiments, concentrated ultrafine bubbles are stable within the composition for at least 2 years.

In some embodiments, the compositions for oral administration and/or ingestion include at least one non-gaseous solute and/or at least one gaseous solute. In some embodiments, the at least one non-gaseous solute and/or at least one gaseous solute includes one or more electrolytes and minerals. In some embodiments, the one or more electrolytes and minerals comprises magnesium, sodium, potassium, chloride, sulfate, benzoate, bicarbonate, zinc, or combinations thereof.

In some embodiments, the composition is a beverage for ingestion. In some embodiments, the beverage is a juice, a still beverage, a carbonated beverage, an energy drink, an electrolyte drink, an alcoholic beverage, a mocktail, coffee, tea, or sweetened, flavored water.

In some embodiments, the beverage for ingestion including ultrafine bubbles comprising or consisting essentially of water and gases released from solution in the water has improved bioavailability relative to naturally occurring water, and relative to compositions including ultrafine bubbles not formed via gaseous cavitation. In some embodiments, the ultrafine bubbles comprising or consisting essentially of water and gases released from solution in the water improve bioavailability of the water by about 10%, about 20%, about 30%, about 40%, about 50%, about 60%, about 70%, about 80%, or about 90% relative to naturally occurring water, and/or relative to compositions including ultrafine bubbles not formed via gaseous cavitation. In further embodiments, the beverage including ultrafine bubbles comprising or consisting essentially of water and gases released from solution in the water improve bioavailability of the water by about 1%, about 2%, about 3%, about 4%, about 5%, about 6%, about 7%, about 8%, or about 9% relative to naturally occurring water, and/or relative to compositions including ultrafine bubbles not formed via gaseous cavitation.

In some embodiments, the beverage for ingestion comprises or consists essentially of at least one non-gaseous solute and/or at least one gaseous solute, water, and ultrafine bubbles comprising water and gases released from solution in the water, and the ultrafine bubbles dissolve/surround/stabilize the at least one solute, thereby improving bioavailability of the at least one solute by about 10%, about 20%, about 30%, about 40%, about 50%, about 60%, about 70%, about 80%, or about 90% relative to the undissolved/unsurrounded/unstabilized solute. In further embodiments, the beverage for ingestion including at least one non-gaseous solute and/or at least one gaseous solute, water, and ultrafine bubbles comprising or consisting essentially of water and gases released from solution in the water improve bioavailability of the at least one solute by about 1%, about 2%, about 3%, about 4%, about 5%, about 6%, about 7%, about 8%, or about 9%.

In some embodiments, the beverage for ingestion comprises at least one non-gaseous solute and/or at least one gaseous solute, wherein the at least one non-gaseous solute and/or at least one gaseous solute is present at a concentration of 0.1-30% by weight of the composition. In other embodiments, the at least one non-gaseous solute and/or at least one gaseous solute is present at a concentration of 0.1-1% by weight of the composition, 1-2% by weight of the composition, 2-3% by weight of the composition, 3-4% by weight of the composition, 4-5% by weight of the composition, 5-6% by weight of the composition, 6-7% by weight of the composition, 7-8% by weight of the composition, 8-9% by weight of the composition, 9-10% by weight of the composition, 10-11% by weight of the composition, 11-12% by weight of the composition, 12-13% by weight of the composition, 13-14% by weight of the composition, 14-15% by weight of the composition, 15-16% by weight of the composition, 16-17% by weight of the composition, 17-18% by weight of the composition, 18-19% by weight of the composition, 19-20% by weight of the composition, 20-21% by weight of the composition, 21-22% by weight of the composition, 22-23% by weight of the composition, 23-24% by weight of the composition, 24-25% by weight of the composition, 25-26% by weight of the composition, 26-27% by weight of the composition, 27-28% by weight of the composition, 28-29% by weight of the composition, or 29-30% by weight of the composition.

In some embodiments, the at least one non-gaseous solute and/or at least one gaseous solute is present in the beverage for ingestion at a low concentration of 0.1-10% by weight of the composition. In other embodiments, the at least one non-gaseous solute and/or at least one gaseous solute is present at a concentration of 0.1-0.5% by weight of the composition, 0.5-1% by weight of the composition, 1-1.5% by weight of the composition, 1.5-2% by weight of the composition, 2-2.5% by weight of the composition, 2.5-3% by weight of the composition, 3-3.5% by weight of the composition, 3.5-4% by weight of the composition, 4-4.5% by weight of the composition, 4.5-5% by weight of the composition, 5-5.5% by weight of the composition, 5.5-6% by weight of the composition, 6-6.5% by weight of the composition, 6.5-7% by weight of the composition, 7-7.5% by weight of the composition, 7.5-8% by weight of the composition, 8-8.5% by weight of the composition, 8.5-9% by weight of the composition, 9-9.5% by weight of the composition, or 9.5-10% by weight of the composition.

In some embodiments, the at least one non-gaseous solute and/or at least one gaseous solute is present in the beverage for ingestion at a medium concentration of 10-30% by weight of the composition. In other embodiments, the at least one non-gaseous solute and/or at least one gaseous solute is present at a concentration of 10-10.5% by weight of the composition, 10.5-11% by weight of the composition, 11-11.5% by weight of the composition, 11.5-12% by weight of the composition, 12-12.5% by weight of the composition, 12.5-13% by weight of the composition, 13-13.5% by weight of the composition, 13.5-14% by weight of the composition, 14-14.5% by weight of the composition, 14.5-15% by weight of the composition, 15-15.5% by weight of the composition, 15.5-16% by weight of the composition, 16-16.5% by weight of the composition, 16.5-17% by weight of the composition, 17-17.5% by weight of the composition, 17.5-18% by weight of the composition, 18-18.5% by weight of the composition, 18.5-19% by weight of the composition, 19-19.5% by weight of the composition, 19.5-20% by weight of the composition, 20-20.5% by weight of the composition, 20.5-21% by weight of the composition, 21-21.5% by weight of the composition, 21.5-22% by weight of the composition, 22-22.5% by weight of the composition, 22.5-23% by weight of the composition, 23-23.5% by weight of the composition, 23.5-24% by weight of the composition, 24-24.5% by weight of the composition, 24.5-25% by weight of the composition, 25-25.5% by weight of the composition, 25.5-26% by weight of the composition, 26-26.5% by weight of the composition, 26.5-27% by weight of the composition, 27-27.5% by weight of the composition, 27.5-28% by weight of the composition, 28-28.5% by weight of the composition, 28.5-29% by weight of the composition, 29-29.5% by weight of the composition, or 29.5-30% by weight of the composition.

In some embodiments, the at least one non-gaseous solute and/or at least one gaseous solute is present in the beverage for ingestion at a high concentration of 30-95% by weight of the composition. In other embodiments, the at least one non-gaseous solute and/or at least one gaseous solute is present at a concentration of 30-35% by weight of the composition, 35-40% by weight of the composition, 40-45% by weight of the composition, 45-50% by weight of the composition, 50-55% by weight of the composition, 55-60% by weight of the composition, 60-65% by weight of the composition, 65-70% by weight of the composition, 70-75% by weight of the composition, 75-80% by weight of the composition, 80-85% by weight of the composition, 85-90% by weight of the composition, or 90-95% by weight of the composition.

In some embodiments, the beverage for ingestion includes one or more flavoring agents. In particular embodiments, the one or more flavoring agents comprise one or more salts. In certain embodiments, the one or more salts comprise at least one of magnesium chloride, calcium chloride, and sodium bicarbonate. In particular embodiments, the one or more flavoring agents is a pungency enhancer. In certain embodiments, the pungency enhancer includes one or more agents derived from black pepper including piperine, chavicine, isopiperine, isochavicine, dihydropiperine, and combinations thereof. In certain embodiments, the one or more flavoring agents includes one or more sweeteners. In certain embodiments, the one or more sweeteners includes nitrous oxide.

In some embodiments, the beverage for ingestion includes one or more cooling-sensation agents. In particular embodiments, the one or more cooling-sensation agents includes one or more of menthol, menthyl lactate, WS-3, WS-23, menthyl carboxamides, and combinations thereof.

In some embodiments, the beverage for ingestion includes one or more warming-sensation agents. In particular embodiments, the one or more warming-sensation agents includes one or more of capsaicin, camphor, eugenol, sanshools, and combinations thereof.

In some embodiments, the beverage for ingestion includes one or more nutrients. In particular embodiments, the one or more nutrients includes dietary collagen. In some embodiments, the at least one non-gaseous solute includes at least one of maltodextrin, vitamin c, gum acacia, niacinamide, monkfruit extract, and zinc sulfate.

In some embodiments, the beverage for ingestion comprises 5 wt % or less of ethanol.

In another aspect, a method for enhancing and/or extending duration of a cooling sensation imparted by a beverage for ingestion in accordance with the disclosure herein is provided. The method comprises ingesting the beverage comprising the ultrafine bubbles. In some embodiments, the cooling sensation imparted by the beverage composition is enhanced in comparison to beverages lacking the ultrafine bubbles. In some embodiments, the cooling sensation imparted by the beverage composition is extended in duration in comparison to beverages lacking the ultrafine bubbles.

In another aspect, a method of enhancing and/or simulating the presence of ethanol within a beverage composition is provided. The method comprises ingesting a beverage composition comprising 5 wt % or less of ethanol and ultrafine bubbles in accordance with the disclosure herein. In some embodiments, the presence of ethanol is enhanced and/or simulated by the ultrafine bubbles of the beverage composition imparting a cooling sensation to the tongue to simulate ethanol evaporation from a tongue. In some embodiments, the presence of ethanol is enhanced and/or simulated by the ultrafine bubbles of the beverage composition increasing the sensory effect of the pungency enhancer to simulate alcohol burn sensation. In particular embodiments, the alcohol burn sensation imparted by the beverage composition is enhanced in comparison to beverages lacking the ultrafine bubbles.

In another aspect, a method for enhancing and/or extending duration of a cooling sensation imparted by a beverage composition is provided. The method includes ingesting the beverage composition comprising the ultrafine bubbles in accordance with the disclosure herein and one or more cooling-sensation agents. In some embodiments, the cooling sensation imparted by the composition is enhanced in comparison to beverages lacking the ultrafine bubbles.

In another aspect, a method for enhancing and/or extending duration of a warming sensation imparted by a beverage composition is provided. The method includes ingesting the beverage composition comprising the ultrafine bubbles in accordance with the disclosure herein and one or more warming-sensation agents. In some embodiments, the warming sensation imparted by the composition is enhanced in comparison to beverages lacking the ultrafine bubbles.

In another aspect, a method for enhancing, increasing, and/or retaining hydration in a subject is provided. The method comprises ingesting a beverage composition comprising the ultrafine bubbles in accordance with the disclosure herein. In some embodiments, the subject is a mammal. In particular embodiments, the subject is a human. In some embodiments, the subject ingests the composition prior to physical exertion. In some embodiments, the subject ingests the composition during physical exertion. In some embodiments, the subject ingests the composition after physical exertion. In some embodiments, the subject experiences a greater decrease in blood serum osmolality after ingesting the composition as compared to after ingesting beverages lacking the ultrafine bubbles. In some embodiments, the subject experiences a decrease in blood serum osmolality for at least 2 hours post physical exertion. In some embodiments, the subject experiences a greater increase in total body water (TBW) after ingesting the composition as compared to ingesting beverages lacking the ultrafine bubbles. In some embodiments, the subject experiences an increase in total body water (TBW) for at least 2 hours post physical exertion. In some embodiments, the subject experiences a greater increase in intracellular water (ICW) after ingesting the composition as compared to ingesting beverages lacking the ultrafine bubbles. In some embodiments, the subject experiences an increase in intracellular water (ICW) for at least 2 hours post physical exertion. In some embodiments, the subject experiences a more rapid increase in intracellular water (ICW) after ingesting the composition as compared to ingesting beverages lacking the ultrafine bubbles.

In another aspect, a method for enhancing absorption of and/or bioavailability of dietary collagen in a subject is provided. The method comprises ingesting a beverage composition comprising dietary collagen as a non-gaseous solute (e.g., a nutrient) and the ultrafine bubbles in accordance with the disclosure herein. In some embodiments, the subject is a mammal. In particular embodiments, the subject is human. In some embodiments, the subject experiences a greater increase in skin resiliency and/or elasticity after ingesting the beverage composition as compared to ingesting beverages including the dietary collagen but lacking the ultrafine bubbles.

In some embodiments, the composition is an oral care solution for oral administration. In some embodiments, the oral care solution is a toothpaste or mouthwash.

In some embodiments, the oral care solution including ultrafine bubbles comprising or consisting essentially of water and gases released from solution in the water has improved bioavailability relative to naturally occurring water, and relative to compositions including ultrafine bubbles not formed via gaseous cavitation. In some embodiments, the ultrafine bubbles comprising or consisting essentially of water and gases released from solution in the water improve bioavailability of the water by about 10%, about 20%, about 30%, about 40%, about 50%, about 60%, about 70%, about 80%, or about 90% relative to naturally occurring water, and/or relative to compositions including ultrafine bubbles not formed via gaseous cavitation. In further embodiments, the oral care solution including ultrafine bubbles comprising or consisting essentially of water and gases released from solution in water improve bioavailability of the water by about 1%, about 2%, about 3%, about 4%, about 5%, about 6%, about 7%, about 8%, or about 9% relative to naturally occurring water, and/or relative to compositions including ultrafine bubbles not formed via gaseous cavitation.

In some embodiments, the oral care solution comprises or consists essentially of at least one non-gaseous solute and/or at least one gaseous solute, water, and ultrafine bubbles comprising water and gases released from solution in the water, and the ultrafine bubbles dissolve/surround/stabilize the at least one solute, thereby improving bioavailability of the at least one solute by about 10%, about 20%, about 30%, about 40%, about 50%, about 60%, about 70%, about 80%, or about 90% relative to the undissolved/unsurrounded/unstabilized solute. In further embodiments, the oral care solution including at least one non-gaseous solute and/or at least one gaseous solute, water, and ultrafine bubbles comprising or consisting essentially of water and gases released from solution in the water improve bioavailability of the at least one solute by about 1%, about 2%, about 3%, about 4%, about 5%, about 6%, about 7%, about 8%, or about 9%.

In some embodiments, the oral care solution includes one or more cooling-sensation agents. In particular embodiments, the one or more cooling-sensation agents includes one or more of menthol, menthyl lactate, WS-3, WS-23, menthyl carboxamides, and combinations thereof.

In some embodiments, the oral care solution includes one or more fluoride sources. In some embodiments, the one or more fluoride sources includes sodium fluoride, stannous fluoride, acidulated phosphate fluoride, and combinations thereof.

In some embodiments, the oral care solution includes one or more antibacterial agents. In particular embodiments, the one or more antibacterial agents includes one or more of menthol, thymol, eucalyptol, methyl salicylate, and combinations thereof.

In some embodiments, the oral care solution comprises at least one non-gaseous solute and/or at least one gaseous solute, wherein the at least one non-gaseous solute and/or at least one gaseous solute is present at a concentration of 0.1-30% by weight of the composition. In other embodiments, the at least one non-gaseous solute and/or at least one gaseous solute is present at a concentration of 0.1-1% by weight of the composition, 1-2% by weight of the composition, 2-3% by weight of the composition, 3-4% by weight of the composition, 4-5% by weight of the composition, 5-6% by weight of the composition, 6-7% by weight of the composition, 7-8% by weight of the composition, 8-9% by weight of the composition, 9-10% by weight of the composition, 10-11% by weight of the composition, 11-12% by weight of the composition, 12-13% by weight of the composition, 13-14% by weight of the composition, 14-15% by weight of the composition, 15-16% by weight of the composition, 16-17% by weight of the composition, 17-18% by weight of the composition, 18-19% by weight of the composition, 19-20% by weight of the composition, 20-21% by weight of the composition, 21-22% by weight of the composition, 22-23% by weight of the composition, 23-24% by weight of the composition, 24-25% by weight of the composition, 25-26% by weight of the composition, 26-27% by weight of the composition, 27-28% by weight of the composition, 28-29% by weight of the composition, or 29-30% by weight of the composition.

In some embodiments, the at least one non-gaseous solute and/or at least one gaseous solute is present in the oral care solution at a low concentration of 0.1-10% by weight of the composition. In other embodiments, the at least one non-gaseous solute and/or at least one gaseous solute is present at a concentration of 0.1-0.5% by weight of the composition, 0.5-1% by weight of the composition, 1-1.5% by weight of the composition, 1.5-2% by weight of the composition, 2-2.5% by weight of the composition, 2.5-3% by weight of the composition, 3-3.5% by weight of the composition, 3.5-4% by weight of the composition, 4-4.5% by weight of the composition, 4.5-5% by weight of the composition, 5-5.5% by weight of the composition, 5.5-6% by weight of the composition, 6-6.5% by weight of the composition, 6.5-7% by weight of the composition, 7-7.5% by weight of the composition, 7.5-8% by weight of the composition, 8-8.5% by weight of the composition, 8.5-9% by weight of the composition, 9-9.5% by weight of the composition, or 9.5-10% by weight of the composition.

In some embodiments, the at least one non-gaseous solute and/or at least one gaseous solute is present in the oral care solution at a medium concentration of 10-30% by weight of the composition. In other embodiments, the at least one non-gaseous solute and/or at least one gaseous solute is present at a concentration of 10-10.5% by weight of the composition, 10.5-11% by weight of the composition, 11-11.5% by weight of the composition, 11.5-12% by weight of the composition, 12-12.5% by weight of the composition, 12.5-13% by weight of the composition, 13-13.5% by weight of the composition, 13.5-14% by weight of the composition, 14-14.5% by weight of the composition, 14.5-15% by weight of the composition, 15-15.5% by weight of the composition, 15.5-16% by weight of the composition, 16-16.5% by weight of the composition, 16.5-17% by weight of the composition, 17-17.5% by weight of the composition, 17.5-18% by weight of the composition, 18-18.5% by weight of the composition, 18.5-19% by weight of the composition, 19-19.5% by weight of the composition, 19.5-20% by weight of the composition, 20-20.5% by weight of the composition, 20.5-21% by weight of the composition, 21-21.5% by weight of the composition, 21.5-22% by weight of the composition, 22-22.5% by weight of the composition, 22.5-23% by weight of the composition, 23-23.5% by weight of the composition, 23.5-24% by weight of the composition, 24-24.5% by weight of the composition, 24.5-25% by weight of the composition, 25-25.5% by weight of the composition, 25.5-26% by weight of the composition, 26-26.5% by weight of the composition, 26.5-27% by weight of the composition, 27-27.5% by weight of the composition, 27.5-28% by weight of the composition, 28-28.5% by weight of the composition, 28.5-29% by weight of the composition, 29-29.5% by weight of the composition, or 29.5-30% by weight of the composition.

In some embodiments, the at least one non-gaseous solute and/or at least one gaseous solute is present in the oral care solution at a high concentration of 30-95% by weight of the composition. In other embodiments, the at least one non-gaseous solute and/or at least one gaseous solute is present at a concentration of 30-35% by weight of the composition, 35-40% by weight of the composition, 40-45% by weight of the composition, 45-50% by weight of the composition, 50-55% by weight of the composition, 55-60% by weight of the composition, 60-65% by weight of the composition, 65-70% by weight of the composition, 70-75% by weight of the composition, 75-80% by weight of the composition, 80-85% by weight of the composition, 85-90% by weight of the composition, or 90-95% by weight of the composition.

In another aspect, a method for enhancing and/or extending duration of a cooling sensation imparted by an oral care composition is provided. The method includes orally administering the oral care composition comprising the ultrafine bubbles in accordance with the disclosure herein and one or more cooling-sensation agents. In some embodiments, the cooling sensation imparted by the oral care composition is enhanced in comparison to oral care compositions including the one or more cooling-sensation agents but lacking the ultrafine bubbles.

In another aspect, a method for enhancing antibacterial efficacy imparted by an oral care composition is provided. The method comprises ingesting the oral care composition comprising the ultrafine bubbles in accordance with the disclosure herein and the one or more antibacterial agents. In some embodiments, the antibacterial efficacy imparted by the oral care composition is enhanced in comparison to oral care compositions including the one or more antibacterial agents but lacking the ultrafine bubbles.

In another aspect, a method for increasing the delivery of fluoride to a subject is provided. The method comprises orally administering an oral care composition including the ultrafine bubbles in accordance with the disclosure herein and one or more fluoride sources to the subject. In some embodiments, the one or more fluoride sources includes sodium fluoride, stannous fluoride, acidulated phosphate fluoride, and combinations thereof. In some embodiments, the subject is a mammal. In particular embodiments, the mammal is a human. In some embodiments, the delivery of fluoride to the subject imparted by the oral care composition comprising the ultrafine bubbles and one or more fluoride sources is increased in comparison to delivery of fluoride by compositions including the one or more fluoride sources but lacking the ultrafine bubbles.

In some embodiments, the composition is an ingestible food product. In some embodiments, the ingestible food product includes one or more flavoring agents as at least one non-gaseous solute. In some embodiments, the one or more flavoring agents is a pungency enhancer. In particular embodiments, the pungency enhancer includes one or more agents derived from black pepper including piperine, chavicine, isopiperine, isochavicine, dihydropiperine, and combinations thereof. In some embodiments, the one or more flavoring agents is a sweetener. In some embodiments, the one or more sweeteners is nitrous oxide.

In some embodiments, the ingestible food product including ultrafine bubbles comprising or consisting essentially of water and gases released from solution in the water has improved bioavailability relative to naturally occurring water, and relative to compositions including ultrafine bubbles not formed via gaseous cavitation. In some embodiments, the ultrafine bubbles comprising or consisting essentially of water and gases released from solution in the water improve bioavailability of the water by about 10%, about 20%, about 30%, about 40%, about 50%, about 60%, about 70%, about 80%, or about 90% relative to naturally occurring water, and/or relative to compositions including ultrafine bubbles not formed via gaseous cavitation. In further embodiments, the ingestible food product including ultrafine bubbles comprising or consisting essentially of water and gases released from solution in water improve bioavailability of the water by about 1%, about 2%, about 3%, about 4%, about 5%, about 6%, about 7%, about 8%, or about 9% relative to naturally occurring water, and/or relative to compositions including ultrafine bubbles not formed via gaseous cavitation.

In some embodiments, the ingestible food product comprises or consists essentially of at least one non-gaseous solute and/or at least one gaseous solute, water, and ultrafine bubbles comprising water and gases released from solution in the water, and the ultrafine bubbles dissolve/surround/stabilize the at least one solute, thereby improving bioavailability of the at least one solute by about 10%, about 20%, about 30%, about 40%, about 50%, about 60%, about 70%, about 80%, or about 90% relative to the undissolved/unsurrounded/unstabilized solute. In further embodiments, the ingestible food product including at least one non-gaseous solute and/or at least one gaseous solute, water, and ultrafine bubbles comprising or consisting essentially of water and gases released from solution in the water improve bioavailability of the at least one solute by about 1%, about 2%, about 3%, about 4%, about 5%, about 6%, about 7%, about 8%, or about 9%.

In some embodiments, the ingestible food product comprises at least one non-gaseous solute and/or at least one gaseous solute, wherein the at least one non-gaseous solute and/or at least one gaseous solute is present at a concentration of 0.1-30% by weight of the composition. In other embodiments, the at least one non-gaseous solute and/or at least one gaseous solute is present at a concentration of 0.1-1% by weight of the composition, 1-2% by weight of the composition, 2-3% by weight of the composition, 3-4% by weight of the composition, 4-5% by weight of the composition, 5-6% by weight of the composition, 6-7% by weight of the composition, 7-8% by weight of the composition, 8-9% by weight of the composition, 9-10% by weight of the composition, 10-11% by weight of the composition, 11-12% by weight of the composition, 12-13% by weight of the composition, 13-14% by weight of the composition, 14-15% by weight of the composition, 15-16% by weight of the composition, 16-17% by weight of the composition, 17-18% by weight of the composition, 18-19% by weight of the composition, 19-20% by weight of the composition, 20-21% by weight of the composition, 21-22% by weight of the composition, 22-23% by weight of the composition, 23-24% by weight of the composition, 24-25% by weight of the composition, 25-26% by weight of the composition, 26-27% by weight of the composition, 27-28% by weight of the composition, 28-29% by weight of the composition, or 29-30% by weight of the composition.

In some embodiments, the at least one non-gaseous solute and/or at least one gaseous solute is present in the ingestible food product at a low concentration of 0.1-10% by weight of the composition. In other embodiments, the at least one non-gaseous solute and/or at least one gaseous solute is present at a concentration of 0.1-0.5% by weight of the composition, 0.5-1% by weight of the composition, 1-1.5% by weight of the composition, 1.5-2% by weight of the composition, 2-2.5% by weight of the composition, 2.5-3% by weight of the composition, 3-3.5% by weight of the composition, 3.5-4% by weight of the composition, 4-4.5% by weight of the composition, 4.5-5% by weight of the composition, 5-5.5% by weight of the composition, 5.5-6% by weight of the composition, 6-6.5% by weight of the composition, 6.5-7% by weight of the composition, 7-7.5% by weight of the composition, 7.5-8% by weight of the composition, 8-8.5% by weight of the composition, 8.5-9% by weight of the composition, 9-9.5% by weight of the composition, or 9.5-10% by weight of the composition.

In some embodiments, the at least one non-gaseous solute and/or at least one gaseous solute is present in the ingestible food product at a medium concentration of 10-30% by weight of the composition. In other embodiments, the at least one non-gaseous solute and/or at least one gaseous solute is present at a concentration of 10-10.5% by weight of the composition, 10.5-11% by weight of the composition, 11-11.5% by weight of the composition, 11.5-12% by weight of the composition, 12-12.5% by weight of the composition, 12.5-13% by weight of the composition, 13-13.5% by weight of the composition, 13.5-14% by weight of the composition, 14-14.5% by weight of the composition, 14.5-15% by weight of the composition, 15-15.5% by weight of the composition, 15.5-16% by weight of the composition, 16-16.5% by weight of the composition, 16.5-17% by weight of the composition, 17-17.5% by weight of the composition, 17.5-18% by weight of the composition, 18-18.5% by weight of the composition, 18.5-19% by weight of the composition, 19-19.5% by weight of the composition, 19.5-20% by weight of the composition, 20-20.5% by weight of the composition, 20.5-21% by weight of the composition, 21-21.5% by weight of the composition, 21.5-22% by weight of the composition, 22-22.5% by weight of the composition, 22.5-23% by weight of the composition, 23-23.5% by weight of the composition, 23.5-24% by weight of the composition, 24-24.5% by weight of the composition, 24.5-25% by weight of the composition, 25-25.5% by weight of the composition, 25.5-26% by weight of the composition, 26-26.5% by weight of the composition, 26.5-27% by weight of the composition, 27-27.5% by weight of the composition, 27.5-28% by weight of the composition, 28-28.5% by weight of the composition, 28.5-29% by weight of the composition, 29-29.5% by weight of the composition, or 29.5-30% by weight of the composition.

In some embodiments, the at least one non-gaseous solute and/or at least one gaseous solute is present in the ingestible food product at a high concentration of 30-95% by weight of the composition. In other embodiments, the at least one non-gaseous solute and/or at least one gaseous solute is present at a concentration of 30-35% by weight of the composition, 35-40% by weight of the composition, 40-45% by weight of the composition, 45-50% by weight of the composition, 50-55% by weight of the composition, 55-60% by weight of the composition, 60-65% by weight of the composition, 65-70% by weight of the composition, 70-75% by weight of the composition, 75-80% by weight of the composition, 80-85% by weight of the composition, 85-90% by weight of the composition, or 90-95% by weight of the composition.

In another aspect, a method for increasing a pungent sensation imparted by pungency enhancers in an ingestible food product is provided. The method includes ingesting an ingestible food product comprising the ultrafine bubbles in accordance with the disclosure herein and the one or more pungency enhancers. In some embodiments, the sensation of pungency is increased by the ultrafine bubbles of the composition in comparison to compositions lacking the ultrafine bubbles.

In another aspect, the compositions are aqueous compositions for topical application or use. The compositions include water and ultrafine bubbles comprising gases released from solution in the water.

In some embodiments, the water is selected from DI water, ultrapure water, tap water, groundwater, surface water, and reverse osmosis water. In some embodiments, the water has a resistivity between about 17 to about 18.2 meg-ohm cm. In further embodiments, the water has a pH of between about 3 to about 7. In some embodiments, the water has an oxidative reduction potential of about −200 mV to about 800 mV.

In some embodiments, the compositions for topical application including ultrafine bubbles comprising or consisting essentially of water and gases released from solution in the water have improved bioavailability relative to naturally occurring water, and relative to compositions including ultrafine bubbles not formed via gaseous cavitation. In some embodiments, the ultrafine bubbles comprising or consisting essentially of water and gases released from solution in the water improve bioavailability of the water by about 10%, about 20%, about 30%, about 40%, about 50%, about 60%, about 70%, about 80%, or about 90% relative to naturally occurring water, and/or relative to compositions including ultrafine bubbles not formed via gaseous cavitation. In further embodiments, compositions for topical application including ultrafine bubbles comprising or consisting essentially of water and gases released from solution in water improve bioavailability of the water by about 1%, about 2%, about 3%, about 4%, about 5%, about 6%, about 7%, about 8%, or about 9% relative to naturally occurring water, and/or relative to compositions including ultrafine bubbles not formed via gaseous cavitation.

In some embodiments, the compositions for topical application comprise or consist essentially of at least one non-gaseous solute and/or at least one gaseous solute, water, and ultrafine bubbles comprising water and gases released from solution in the water, and the ultrafine bubbles dissolve/surround/stabilize the at least one solute, thereby improving bioavailability of the at least one solute by about 10%, about 20%, about 30%, about 40%, about 50%, about 60%, about 70%, about 80%, or about 90% relative to the undissolved/unsurrounded/unstabilized solute. In further embodiments, the compositions for topical application including at least one non-gaseous solute and/or at least one gaseous solute, water, and ultrafine bubbles comprising or consisting essentially of water and gases released from solution in the water improve bioavailability of the at least one solute by about 1%, about 2%, about 3%, about 4%, about 5%, about 6%, about 7%, about 8%, or about 9%.

In some embodiments, the at least one non-gaseous solute and/or at least one gaseous solute is dissolved within, surrounded by, and/or stabilized by the ultrafine bubbles. In some embodiments, the composition increases cell permeability and/or bioavailability of the at least one dissolved non-gaseous solute. In some embodiments, the at least one non-gaseous solute and/or at least one gaseous solute is present at a concentration of 0.1-30% by weight of the composition. In other embodiments, the at least one non-gaseous solute and/or at least one gaseous solute is present at a concentration of 0.1-1% by weight of the composition, 1-2% by weight of the composition, 2-3% by weight of the composition, 3-4% by weight of the composition, 4-5% by weight of the composition, 5-6% by weight of the composition, 6-7% by weight of the composition, 7-8% by weight of the composition, 8-9% by weight of the composition, 9-10% by weight of the composition, 10-11% by weight of the composition, 11-12% by weight of the composition, 12-13% by weight of the composition, 13-14% by weight of the composition, 14-15% by weight of the composition, 15-16% by weight of the composition, 16-17% by weight of the composition, 17-18% by weight of the composition, 18-19% by weight of the composition, 19-20% by weight of the composition, 20-21% by weight of the composition, 21-22% by weight of the composition, 22-23% by weight of the composition, 23-24% by weight of the composition, 24-25% by weight of the composition, 25-26% by weight of the composition, 26-27% by weight of the composition, 27-28% by weight of the composition, 28-29% by weight of the composition, or 29-30% by weight of the composition.

In some embodiments, the at least one non-gaseous solute and/or at least one gaseous solute is present in the composition for topical application at a low concentration of 0.1-5% by weight of the composition. In other embodiments, the at least one non-gaseous solute and/or at least one gaseous solute is present at a concentration of 0.1-0.5% by weight of the composition, 0.5-1% by weight of the composition, 1-1.5% by weight of the composition, 1.5-2% by weight of the composition, 2-2.5% by weight of the composition, 2.5-3% by weight of the composition, 3-3.5% by weight of the composition, 3.5-4% by weight of the composition, 4-4.5% by weight of the composition, or 4.5-5% by weight of the composition.

In some embodiments, the at least one non-gaseous solute and/or at least one gaseous solute is present in the composition for topical application at a medium concentration of 5-15% by weight of the composition. In other embodiments, the at least one non-gaseous solute and/or at least one gaseous solute is present at a concentration of 5-5.5% by weight of the composition, 5.5-6% by weight of the composition, 6-6.5% by weight of the composition, 6.5-7% by weight of the composition, 7-7.5% by weight of the composition, 7.5-8% by weight of the composition, 8-8.5% by weight of the composition, 8.5-9% by weight of the composition, and 9-9.5% by weight of the composition, 9.5-10% by weight of the composition, 10-10.5% by weight of the composition, 10.5-11% by weight of the composition, 11-11.5% by weight of the composition, 11.5-12% by weight of the composition, 12-12.5% by weight of the composition, 12.5-13% by weight of the composition, 13-13.5% by weight of the composition, 13.5-14% by weight of the composition, 14-14.5% by weight of the composition, or 14.5-15% by weight of the composition.

In some embodiments, the at least one non-gaseous solute and/or at least one gaseous solute is present in the composition for topical application at a high concentration of 15-30% by weight of the composition. In other embodiments, the at least one non-gaseous solute and/or at least one gaseous solute is present at a concentration of 15-15.5% by weight of the composition, 15.5-16% by weight of the composition, 16-16.5% by weight of the composition, 16.5-17% by weight of the composition, 17-17.5% by weight of the composition, 17.5-18% by weight of the composition, 18-18.5% by weight of the composition, 18.5-19% by weight of the composition, and 19-19.5% by weight of the composition, 19.5-20% by weight of the composition, 20-20.5% by weight of the composition, 20.5-21% by weight of the composition, 21-21.5% by weight of the composition, 21.5-22% by weight of the composition, 22-22.5% by weight of the composition, 22.5-23% by weight of the composition, 23-23.5% by weight of the composition, 23.5-24% by weight of the composition, 24-24.5% by weight of the composition, 24.5-25% by weight of the composition, 25-25.5% by weight of the composition, 25.5-26% by weight of the composition, 26-26.5% by weight of the composition, 26.5-27% by weight of the composition, 27-27.5% by weight of the composition, 27.5-28% by weight of the composition, 28-28.5% by weight of the composition, 28.5-29% by weight of the composition, 29-29.5% by weight of the composition, or 29.5-30% by weight of the composition.

In some embodiments of the methods herein, ultrapure water of the disclosure comprises water substantially free of or completely free of contaminants (e.g., an impurity). As used herein, a contaminant is a foreign substance not intentionally added to the ultrapure water produced according to the disclosure. Thus, ultrapure water substantially free of contaminants contains undetectable levels/amounts of, for example, the following contaminants: (a) pathogenic bacteria (e.g., fecal coliform), viruses (e.g., hepatitis viruses, hemorrhagic viruses, retroviruses such as AIDS virus), fungi, mycoplasm, protozoa, prokaryotes, protists, parasites, microorganisms causing infectious diseases, and their spores, eggs, DNA, RNA, or related reproductive constituents, prions, (b) toxic biochemicals including toxic proteins, lipids, carbohydrates, and toxic nucleic acids; (c) toxic inorganic chemicals (soluble and insoluble in water and including toxic heavy metals) and their particles; (d) toxic organic chemicals (soluble and insoluble in water and including pesticides) and their particles; (e) non-water organic liquids (miscible and immiscible); (f) radioactive minerals, or (g) toxic gases including ammonia, arsenic pentafluoride, arsine, bis(trifluoromethyl) peroxide, boron tribromide, boron trichloride, boron trifluoride, bromine, bromine chloride, boromethane, carbon monoxide, chlorine, chlorine pentafluoride, chlorine trifluoride, chloropicrin, cyanogen, cyanogen chloride, diazomethane, diborane, dichloroacetylene, dichlorosilane, fluorine, formaldehyde, germane, hexylethyl tetraphosphate, hydrogen azide, hydrogen cyanide, hydrogen selenide, hydrogen sulfide, hydrogen telluride, nickel tetracarbonyl, nitrogen dioxide, osmium tetroxide, oxygen difluoride, perfluroisobutylene, phosgene, phosphine, phosphorus pentafluoride, selenium hexafluoride, silicon hexafluoride, silicon tetrachloride, stilbene, disulfur decafluoride, sulfur tetrafluoride, tellurium hexafluoride, tetraethyl pyrophosphate, tetraethyl dithiopyrophosphate, trifluoroacetyl chloride, tungsten hexafluroide, and radon.

Ultrapure water of the disclosure may be prepared by processes known in the art and used as a starting material for generating the compositions and solutions comprising ultrafine bubbles as disclosed herein. The ultrapure water of the disclosure may be prepared by carbon filtration, by slow sand filtration, by reverse osmosis, by electro-deionization treatment, by ultraviolet light exposure, or by a combination comprising two or more of the processes described herein. For example, the ultrapure water of the disclosure may be prepared by a sequential process comprising each of carbon filtration, slow sand filtration, reverse osmosis, electro-deionization treatment, and ultraviolet light exposure. Alternatively, the ultrapure water may be prepared according to one or more of the processes described herein in combination with other methods of water purification known in the art but not expressly recited herein.

The ultrapure water may be prepared by a process comprising the steps of: filtering a volume of water with a carbon filter to produce an amount of water with a low chlorine content; removing ions in the carbon filtered water by a reverse osmosis process to produce a supply of a deionized water; electro-deionizing the supply of the deionized water from the reverse osmosis process to make an ultrapure water supply; testing the resistivity of the ultrapure water to determine if the resistivity of the ultrapure water is between about 17 meg-ohm cm to about 18.2 meg-ohm cm; repeating a process step for preparing the ultrapure water and retesting the resistivity of the ultrapure water until the ultrapure water has a measured resistivity of between about 17 meg-ohm cm to about 18.2 meg-ohm cm; irradiating the supply of the ultrapure water having a measured resistivity of between about 17 meg-ohm cm to about 18.2 meg-ohm cm with ultraviolet light to make a sterilized ultrapure water supply; and storing the sterilized ultrapure water in a stainless steel container until sterilized ultrapure water is needed to be added in the process to make an aqueous composition comprising an aqueous medium with reduced size ultrafine bubbles containing a solute to improve bioavailability of the aqueous composition.

The ultrapure water is purified of contaminants including, for example, organic and inorganic compounds; dissolved and particulate matter; volatile and non-volatile matter, reactive and inert matter; and hydrophilic and hydrophobic matter. Ultrapure water and commonly used term deionized (DI) water are not the same. An ultrapure water system may include three stages: a pretreatment stage to produce purified water, a primary stage to further purify the water, and a polishing stage. The most widely used requirements for ultrapure water quality are documented by ASTM D5127 “Standard Guide for Ultra-Pure Water Used in the Electronics and Semiconductor Industries” and SEMI F63 “Guide for ultrapure water used in semiconductor processing.”

The polishing stage may include continuously treating and recirculating the purified water in order to maintain stable high purity quality of supplied water. Traditionally the resistivity of water serves as an indication of the level of purity of ultrapure water. Deionized (DI) water may have a purity of at least one million ohms-centimeter or one meg-ohm cm. In a preferred embodiment, the ultrapure water quality is at the theoretical maximum of water resistivity (18.18 meg-ohm cm at 25° C.).

The ultrapure water of the disclosure may have a high oxidative reduction potential including, for example, about 140 to about 160 mV. Further, the pH of the ultrapure water may be between about 3 to about 7, preferably about 4 to about 6 and the resistivity of the ultrapure water may be between about 17 to about 18.2 meg-ohm cm.

In some embodiments of the methods disclosed herein, the compositions and solutions used in the methods include ultrafine bubbles comprising or consisting essentially of ultrapure water and gases released from solution in the ultrapure water, wherein the ultrafine bubbles dissolve, surround, and/or stabilize a solute, and wherein the ultrapure water has a high negative oxidative reduction potential. In further embodiments, the oxidative reduction potential of the ultrapure water is about 80 mV to about 100 mV, about 100 mV to about 120 mV, about 120 mV to about 140 mV, or about 140 mV to about 160 mV. In still further embodiments, the pH of the ultrapure water is between about 4 to about 5, about 5 to about 6, or about 6 to about 7.

A non-gaseous solute and/or at least one gaseous solute dissolved in a composition including ultrafine bubbles comprising or consisting essentially of water and gases released from solution in the water may have an approximately round geometry, a flat plate geometry, a cube geometry, a rod-like geometry, a hollow geometry, and/or a semi-hollow geometry. In some embodiments, a solute dissolved in a composition including ultrafine bubbles comprising or consisting essentially of water and gases released from solution in the water may comprise a primary solute, a mixture of a first solute and a second solute, or a plurality of solutes. The solutes may have one or more additional associated solutes, such as a surface coating, as a subsurface coating, or in a complex with other solutes. The solutes may comprise a liquid, a solid, a gas, or be a colloidal system with a colloid and a dispersing agent. The solutes may be at least minimally soluble in water, with or without the addition of a surfactant.

In some embodiments of the methods claimed herein, the at least one non-gaseous solute and/or at least one gaseous solute comprises one or more of cooling sensation agents, warming sensation agents, antibacterial agents, pain relief agents, numbing agents, hair restorative agents, anti-itch agents, topical antihistamines, pungency agents, skin hydration agents, anti-acne agents, flavoring agents, nutrients, electrolytes, minerals, alcohols, and fluoride sources. In some embodiments, the cooling sensation agents include at least one of menthol, menthyl lactate, WS-3, WS-23 and other menthyl carboxamides. In some embodiments, the warming sensation agents include at least one of capsaicin, camphor, eugenol, and sanshools. In some embodiments, the pain relief agents include at least one of lidocaine, benzocaine, pramoxine, phenol, and methyl salicylate. In some embodiments, the numbing agents include at least one of lidocaine, benzocaine, pramoxine, phenol, and methyl salicylate. In some embodiments, the hair restorative agents include at least one of minoxidil and finasteride. In some embodiments, the anti-itch agents include at least one of diphenhydramine, azelastine, olopatadine, ketotifen, and hydrocortisone. In some embodiments, the pungency agents include at least one of piperine, chavicine, isopiperine, dihydropiperine, and isochavicine. In some embodiments, the fluoride sources include at least one of sodium fluoride, stannous fluoride, and acidulated phosphate fluoride. In some embodiments, the anti-acne agents include at least one of benzoyl peroxide, salicylic acid, retinoids, topical antibiotics (e.g. clindamycin, erythromycin), azelaic acid, sulfur, alpha hydroxy acids (AHAs), nicotinamide (niacinamide), dapsone, and tea tree oil. In some embodiments, the one or more flavoring agents comprise one or more salts. In particular embodiments, the one or more salts comprise at least one of magnesium chloride, calcium chloride, and sodium bicarbonate. In some embodiments, the at least one non-gaseous solute and/or at least one gaseous solute comprises one or more electrolytes and minerals. In particular embodiments, the one or more electrolytes and minerals comprises magnesium, sodium, potassium, chloride, sulfate, benzoate, bicarbonate, zinc, or combinations thereof.

In some embodiments, the at least one non-gaseous solute and/or at least one gaseous solute comprises one or more of a compound that must penetrate the skin barrier/membrane or skin tissue to be effective. In some embodiments, the at least one non-gaseous solute and/or at least one gaseous solute comprises one or more of a compound with low bioavailability.

The ultrafine bubbles comprising or consisting essentially of water and gases released from solution in the water and have a median diameter of between about 2 to about 400 nanometers or comprise about 10 to about 500 molecules of water per ultrafine bubble. In certain embodiments, the ultrafine bubbles have a median diameter of about 1 nanometer, about 2 nanometers, about 3 nanometers, about 4 nanometers, about 5 nanometers, about 6 nanometers, about 7 nanometers, about 8 nanometers, about 9 nanometers, about 10 nanometers, about 11 nanometers, about 12 nanometers, about 13 nanometers, about 14 nanometers, about 15 nanometers, about 16 nanometers, about 17 nanometers, about 18 nanometers, about 19 nanometers, or about 20 nanometers. In other embodiments, the ultrafine bubbles according to the disclosure comprise a median diameter of about 20 nanometers, about 22 nanometers, about 24 nanometers, about 26 nanometers, about 28 nanometers, or about 30 nanometers. In still other embodiments, the ultrafine bubbles according to the disclosure comprise a median diameter of about 35 nanometers, about 40 nanometers, about 45 nanometers, about 50 nanometers, about 60 nanometers, about 70 nanometers, about 80 nanometers, about 90 nanometers, or about 100 nanometers.

In some embodiments, the ultrafine bubble comprises about 20, about 25, about 30, about 35, about 40, about 45, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, or about 500 water molecules. In other embodiments, the ultrafine bubble comprises between about 50 and about 100 water molecules, about 100 to about 150 water molecules, about 150 to about 200 water molecules, about 200 to about 250 water molecules, about 250 to about 300 water molecules, about 300 to about 350 water molecules, about 350 to about 400 water molecules, about 400 to about 450 water molecules, or about 450 to about 500 water molecules.

In some embodiments, the ultrafine bubbles fully dissolve, surround, and/or stabilize a non-gaseous solute and/or at least one gaseous solute or substantially dissolve, surround, and/or stabilize a non-gaseous solute and/or a gaseous solute (e.g., dissolves, surrounds, and/or stabilizes about 50%, 55%, 60%, 65%, 70%, 80%, 85%, 90%, 95% or more of an individual ion or molecule of the non-gaseous solute and/or the gaseous solute).

Those skilled in the art will recognize different ways of measuring a diameter of an ultrafine bubble of the disclosure. In an exemplary method a diameter of an ultrafine bubble is measured using a Malvern Instruments Zetasizer Nano ZSP, which is a high performance system and particularly suitable for the characterization of ultrafine bubbles, solutes, e.g. proteins and other nanoparticles. Optionally, the particle size measurements for the Zetasizer Nano are automated using a NanoSampler. In another exemplary method a diameter of an ultrafine bubble is measured using liquid-cell transmission electron microscopy (TEM). Additionally, the size distribution and concentration of an ultrafine bubble suspension may be measured on a particle-by-particle basis using tunable resistive pulse sensing (TRPS) or electrical zone sensing, using such instruments as the Izon Exoid or the Beckman Coulter Multisizer 4e, respectively.

In some embodiments, the ultrafine bubble and solutes of the disclosure are measured according to the following non-limiting parameters: ultrafine bubble diameter, particle and molecule size, translational diffusion, electrophoretic mobility, zeta potential of particles at high and low concentrations, viscosity and viscoelasticity of protein and polymer solutions, concentration, and/or molecular weight (e.g. kp).

In some embodiments, the ultrafine bubbles comprising or consisting essentially of water and gases released from solution in the water used in such methods are stable for an extended storage period including, for example, a period of years. In some embodiments, the ultrafine bubbles are stable for about 6 months, 1 year, 2 years, about 4 years, about 6 years, about 8 years, or about 10 years. In some embodiments, the ultrafine bubbles are stable for a period in excess of 10 years.

In some embodiments, the ultrafine bubbles used in embodiments of the methods stabilize, surround, and/or dissolve a non-gaseous solute for a period of years, for example for about 6 months, 1 year, 2 years, about 4 years, about 6 years, about 8 years, or about 10 years. In further embodiments, the ultrafine bubbles dissolve, surround, and/or stabilize a non-gaseous solute for a period in excess of 10 years.

In some embodiments, the compositions or solutions include ultrafine bubbles comprising or consisting essentially of water and gases released from solution in the water, wherein the water has a high negative oxidative reduction potential including, for example, an oxidative reduction potential of about 140 to about 160 mV. In still further embodiments, the pH of the water is between about 4 to about 6. In some embodiments, the water is ultrapure water. In some embodiments, the water is tap water.

In some embodiments, the disclosure provides compositions or solutions for use in delivering a non-gaseous solute and/or a gaseous solute to the interior of a cell. In other embodiments, the disclosure provides compositions or solutions for use in delivering a non-gaseous solute and/or a gaseous solute to the interior of a cell (e.g., an animal cell, a mammalian cell, a human cell).

Embodiments of the methods set forth in the disclosure include compositions or solutions wherein a non-gaseous solute and/or a gaseous solute is dissolved within, surrounded by, and/or stabilized by ultrafine bubbles comprising or consisting essentially of water and gases released from solution in the water, and has improved bioavailability relative to a composition or a solution where the solute is not dissolved, stabilized, and/or surrounded by ultrafine bubbles comprising or consisting essentially of water and gases released from solution in the water. In some embodiments, the solute dissolved within, surrounded by, and/or stabilized by ultrafine bubbles has improved bioavailability by virtue of its ability to access the interior of a cell. In some embodiments, the solute dissolved within, surrounded by, and/or stabilized by an ultrafine bubble has improved bioavailability by virtue of its ability to access the interior of an animal cell. For example, a water may have an impurity or a solute that is typically incapable of passing through a cell membrane, but the solute dissolved within, surrounded by, and/or stabilized by the ultrafine bubbles of the disclosure are able to cross a cell membrane. In some embodiments, a cell membrane may be a plasma membrane, a nuclear membrane, or any other impermeable barrier defining the boundaries of a cell or an organelle within a cell.

In other embodiments of the methods set forth herein, a solute dissolved within, surrounded by, or stabilized by ultrafine bubbles comprising or consisting essentially of water and gases released from solution in the water has improved bioavailability by virtue of its ability to access an intracellular space. In still other embodiments, the solute dissolved within, surrounded by, and/or stabilized by ultrafine bubbles has improved bioavailability by virtue of its ability to access specific animal tissue types, such as skin tissue in an animal. In yet other embodiments, an ultrafine bubble comprising or consisting essentially of ultrapure water and gases released from solution in the water has improved bioavailability relative to an ultrafine bubble that does not comprise ultrapure water.

In some embodiments, the aqueous compositions including ultrafine bubbles comprising or consisting essentially of water and gases released from solution in the water and dissolved/surrounded/stabilized solutes have improved bioavailability relative to naturally occurring water and dissolved solutes, and relative to compositions including ultrafine bubbles not formed via gaseous cavitation. In some embodiments, the ultrafine bubbles and dissolved/surrounded/stabilized solutes provided herein render an otherwise unavailable solute bioavailable, in which case the disclosure provides improved bioavailability of the solute relative to the undissolved/unsurrounded/unstabilized solute. In other embodiment, the ultrafine bubbles comprising or consisting essentially of water and gases released from solution in the water, wherein the ultrafine bubbles dissolve/surround/stabilize a solute improve bioavailability of the solute by about 10%, about 20%, about 30%, about 40%, about 50%, about 60%, about 70%, about 80%, or about 90% relative to the undissolved/unsurrounded/unstabilized solute. In further embodiments, the ultrafine bubbles comprising or consisting essentially of water and dissolved/surrounded/stabilized solutes improve bioavailability of the solute by about 1%, about 2%, about 3%, about 4%, about 5%, about 6%, about 7%, about 8%, or about 9%.

In some embodiments, the disclosure provides methods for improving the bioavailability of a solute, including, for example, adding the solute to water and dissolving, surrounding, and/or stabilizing the solute with ultrafine bubbles, wherein the ultrafine bubbles have a median diameter between about 2 to about 400 nanometers.

In some embodiments, the compositions having a solute dissolved/surrounded/stabilized within ultrafine bubbles comprising or consisting essentially of water and gases released from solution in the water used in the disclosed methods have improved stability relative to compositions having the undissolved/unsurrounded/unstabilized solute. In some embodiments, the solute dissolved/surrounded/stabilized by ultrafine bubbles with improved stability has an increased half-life, such as an increased solution half-life. In some embodiments, the solute dissolved/surrounded/stabilized by ultrafine bubbles comprising or consisting essentially of water and gases released from solution in the water has improved stability for extended storage periods relative to the undissolved/unsurrounded/unstabilized solute.

In some embodiments, the compositions or solutions including ultrafine bubbles comprising or consisting essentially of water and gases released from solution in the water and a solute dissolved/surrounded/stabilized within the ultrafine bubbles used in the disclosed methods have improved solubility relative to compositions or solutions including the undissolved/unstabilized/unsurrounded solute.

In some embodiments, the solute dissolved/stabilized within an ultrafine bubble normally has limited or no solubility in water but is solubilized when dissolved/stabilized in ultrafine bubbles comprising or consisting essentially of water and gases released from solution in the water. In alternative embodiments, the solute dissolved/stabilized/surrounded by ultrafine bubbles may have low to moderate solubility in water but is solubilized (e.g., completely solubilized) when dissolved/stabilized/surrounded by ultrafine bubbles comprising or consisting essentially of water and gases released from solution in the water as set forth herein.

In some embodiments, a solute of the disclosure further comprises a surface coating applied before or after dissolving/stabilizing/surrounding the solute with ultrafine bubbles comprising or consisting essentially of water and gases released from solution in the water. For biological applications, such as proteins, the surface coating may be polar to give high aqueous solubility and prevent particle aggregation.

The present disclosure also provides methods for dissolving/surrounding/stabilizing a solute with ultrafine bubbles comprising or consisting essentially of water.

In some embodiments, the disclosure provides a process for dissolving, surrounding, and/or stabilizing a non-gaseous solute and/or a gaseous solute with ultrafine bubbles comprising or consisting essentially of water and gases released from solution in the water, the process comprising: selecting an amount of solute to add to a volume of water; combining the solute and water in a mixing tank to form a blended aqueous composition; pumping the blended aqueous composition at a selected flow rate through a transfer pipe from the mixing tank to a nozzle with one jet opening or a plurality of jet openings inside a hollow cylinder; using the one jet opening or the plurality of jet openings in the nozzle to jet the blended aqueous composition into the hollow cylinder; wherein the selected flow rate creates a vortex of the blended aqueous composition inside the hollow cylinder that dissolve, surround, and/or stabilizes the solutes and reduce sizes of the ultrafine bubbles in the blended aqueous composition. The process according to certain embodiments may further comprise collecting the composition comprising the solute dissolved within, surrounded by, and/or stabilized by the ultrafine bubbles; and using the reduced size ultrafine bubbles dissolving, surrounding, and/or stabilizing the solute to improve the bioavailability of the solute.

In some embodiments, a process is provided for reducing the size of ultrafine bubbles in a solution of water substantially free of dissolved non-gaseous solutes and/or gaseous solutes comprising pumping water at a selected flow rate through a transfer pipe to a nozzle with one jet opening or a plurality of jet openings inside a hollow cylinder; using the one jet opening or the plurality of jet openings in the nozzle to jet the blended composition into the hollow cylinder; wherein the selected flow rate creates a vortex of the blended composition inside the hollow cylinder that dissolve, surround, and/or stabilizes the solutes and reduce the size of the ultrafine bubbles in the blended composition.

In another aspect of the invention disclosed herein, a method for producing a composition comprising water and ultrafine bubbles including gases released from solution in the water is provided. The method includes subjecting water to a combination of hydrodynamic cavitation, shear forces, and low pressure/room temperature boiling to produce ultrafine bubbles formed by release of dissolved gases from the water. In some embodiments, the water is selected from DI water, ultrapure water, tap water, groundwater (e.g., well water), surface water, and reverse osmosis water. In particular embodiments, the water is ultrapure water.

The method may comprise one or more (including all) of the following steps: adding water to a tank; pumping the water at a selected flow rate through a transfer pipe from the tank to a nozzle with one jet opening or a plurality of jet openings inside a hollow cylinder; using the one jet opening or the plurality of jet openings in the nozzle to jet the water into the hollow cylinder; wherein the selected flow rate creates a vortex of the water inside the hollow cylinder, thereby subjecting the water to a combination of hydrodynamic cavitation, shear forces, and thin film boiling to produce ultrafine bubbles formed by release of dissolved gases from the water (i.e., gaseous cavitation); collecting the composition comprising the water and ultrafine bubbles; adding a non-gaseous solute and/or a gaseous solute (e.g., a plant nutrient) to the composition comprising the water and ultrafine bubbles; and using the ultrafine bubbles of the composition to dissolve, surround, and/or stabilized a non-gaseous solute and/or a gaseous solute to improve the bioavailability of the solute.

In some embodiments, a water supply is subjected to a combination of hydrodynamic cavitation, shear forces, and low pressure/room temperature boiling to form ultrafine bubbles, and the formed ultrafine bubbles from the water supply are added to water to form the composition. In some embodiments, a water supply is subjected to processing that forms ultrafine bubbles via gaseous cavitation, and the formed ultrafine bubbles from the water supply are added to water to form a composition as set forth herein. In other embodiments, ultrafine bubbles comprising water and gases released from solution in a first water source are added to a second water source to make compositions as set forth herein. In some embodiments, a non-gaseous solute and/or a gaseous solute is added to the composition including the formed ultrafine bubbles and the water supply to dissolve, surround, and/or stabilize the non-gaseous solute and/or the gaseous solute with the formed ultrafine bubbles.

The present disclosure also provides methods for dissolving, surrounding, and/or stabilizing a non-gaseous solute and/or a gaseous solute with ultrafine bubbles comprising or consisting essentially of water and gases released from solution in water.

In some embodiments, the disclosure provides a process for dissolving, surrounding, and/or stabilizing a non-gaseous solute and/or a gaseous solute with ultrafine bubbles comprising or consisting essentially of water and gases released from solution in water, the process comprising: adding a non-gaseous solute and/or a gaseous solute (e.g., a plant nutrient) to a composition comprising the water and ultrafine bubbles formed by release of dissolved gases from the water; and using the ultrafine bubbles of the composition to dissolve, surround, and/or stabilized a non-gaseous solute and/or a gaseous solute. The process according to certain embodiments may further comprise collecting the composition comprising the non-gaseous solute and/or the gaseous solute dissolved within, surrounded by, and/or stabilized by the ultrafine bubbles; and using the reduced size ultrafine bubbles dissolving, surrounding, and/or stabilizing the non-gaseous solute and/or the gaseous solute to improve the bioavailability of the solute.

In some embodiments, the disclosure provides a process for dissolving, surrounding, and/or stabilizing a non-gaseous solute and/or a gaseous solute with ultrafine bubbles comprising or consisting essentially of water and gases released from solution in water, the process comprising: selecting an amount of solute to add to a volume of water; combining the solute and water in a mixing tank to form a blended aqueous composition; pumping a volume of water at a selected flow rate through a transfer pipe from the mixing tank to a nozzle with one jet opening or a plurality of jet openings inside a hollow cylinder; using the one jet opening or the plurality of jet openings in the nozzle to jet the volume of water into the hollow cylinder; wherein the selected flow rate creates a vortex of the volume of water inside the hollow cylinder that dissolve, surround, and/or stabilizes the solutes and reduce sizes of the ultrafine bubbles in the blended aqueous composition. The process according to certain embodiments may further comprise collecting the composition comprising the solute dissolved within, surrounded by, and/or stabilized by the ultrafine bubbles; and using the reduced size ultrafine bubbles dissolving, surrounding, and/or stabilizing the solute to improve the bioavailability of the solute.

In some embodiments, a process is provided for reducing the size of ultrafine bubbles in a solution of water substantially free of dissolved non-gaseous solutes and/or gaseous solutes comprising pumping water at a selected flow rate through a transfer pipe to a nozzle with one jet opening or a plurality of jet openings inside a hollow cylinder; using the one jet opening or the plurality of jet openings in the nozzle to jet the blended composition into the hollow cylinder; wherein the selected flow rate creates a vortex of the blended composition inside the hollow cylinder that reduces the size of the ultrafine bubbles in the blended composition.

In some embodiments of the methods, the methods further comprise concentrating the ultrafine bubbles within the composition via rotary evaporation or cross flow filtration. This disclosure is further illustrated by the following examples, which are provided to facilitate the practice of the disclosed methods. These examples do not limit the scope of the disclosure in any way.

### EXAMPLES

### Example 1: Methods of Making Compositions Including Water and Ultrafine Bubbles

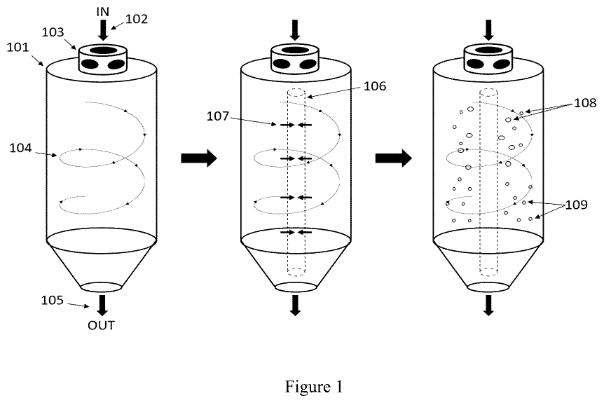

With reference to FIG. 1, a system (101) and a process for making compositions including water and ultrafine bubbles in accordance with embodiments of the invention is provided. Water enters the system (101) at step (102) via the nozzle (103) and imparts a vortex flow (104). The vortex core (106) forms as dissolved gases are drawn out of solution due to low pressure at the center (107). Without being bound by theory, it is believed micro- and ultrafine bubbles form (108) spontaneously due to low pressures near to core surface, due to gas being sheared from the core surface, and/or due to room-temperature low pressure boiling at the core surface. Shear and drag forces are believed to break the microbubbles into ultrafine bubbles resulting in a near uniform size distribution (109). The resulting composition including water and ultrafine bubbles flows from the system (101) via the exit (105).

In an embodiment of the invention, a non-gaseous solute (e.g., a plant nutrient) is added to the water prior to its entry to the system 101 at step 102, and the resulting composition including water, ultrafine bubbles, and the non-gaseous solute (e.g., a plant nutrient) flows from the system (101) via the exit (105). The ultrafine bubbles of the composition dissolve, surround, and/or stabilize the non-gaseous solute.

In another embodiment of the invention, a non-gaseous solute (e.g., a plant nutrient) is added to a composition including water and ultrafine bubbles after the composition exits from the system (101) via the exit (105). The ultrafine bubbles of the composition dissolve, surround, and/or stabilize the non-gaseous solute.

In another embodiment of the invention, the system (101) is used to produce an ultrafine bubble suspension or composition comprising water and ultrafine bubbles. The ultrafine bubbles from the ultrafine bubble suspension or composition comprising water and ultrafine bubbles are then added to a different source of water to form a second composition. A non-gaseous solute (e.g., a plant nutrient) is the added to the second composition. The ultrafine bubbles of the second composition dissolve, surround, and/or stabilize the non-gaseous solute.

In another embodiment of the invention, the water is “enriched” with microbubbles (bubbles greater than one micron and less than a millimeter in diameter) prior to entering the system (101) via the nozzle (103). These bubbles are added from an exogenous source such as a microbubble generator, venturi, or porous bubbler/membrane in-line or into a tank before processing. The resulting composition exiting via the exit (105) may have higher concentrations of ultrafine bubbles as a result (e.g., greater than 10⁷ ultrafine bubbles/mL). This is due to the breakup of the microbubbles into ultrafine bubbles while passing through the system during which, the microbubbles are exposed to drag forces. Furthermore, by creating microbubbles from specific gases, particularly gases that do not readily dissolve into water (e.g. ozone), the composition of the resulting ultrafine bubbles may be controlled or tailored to include a wider range of gases.

In another embodiment of the invention, the water is sparged with one or more specific gases prior to entering the system (101) via the nozzle (103). In some embodiments, the resulting composition of gases contained within the ultrafine bubbles is tailored. For example, when O₂ gas and N₂ gas are sparged or bubbled in water in order to saturate the water prior to undergoing the process within system 101, the resulting composition will have a higher concentration of O₂ and N₂ ultrafine bubbles than if the water had only been exposed to the atmosphere.

### Example 2: Water Composition Hydration Over Time Study

Red blood cells (RBCs) are sensitive to osmotic change and can serve as models for hydration ability, as they release Heme upon lysis (which may occur as a result of rapid increase in hydration). Water compositions were prepared and assessed for ability to hydrate red blood cells in vitro. Three water compositions were prepared with added electrolytes in concentrations as shown in Table 1:
Composition 1) a reverse osmosis (RO) water with added electrolytes (RO w/Macro);
Composition 2) a deionized (DI) water composition prepared according to the disclosure, wherein the electrolytes were added after the water composition was prepared according to the disclosure (EDI Macro #2); and
Composition 3) a deionized (DI) water composition prepared according to the disclosure, wherein the electrolytes were added prior to preparing the water composition according to the disclosure (EDI Tech #2).

TABLE 1
Electrolyte composition added to water compositions:

| | Conc |
|---|---|
| Ion | [mg/L] |
| Magnesium | 22.6 |
| Sodium | 155.5 |
| Potassium | 9.6 |
| Chloride | 65.6 |
| Sulfate | 3.0 |
| Benzoate | 62.9 |
| Bicarbonate | 362.6 |

1 mL of human whole blood K2EDTA was lysed with 1% Triton X-100 and centrifuged to collect the supernatant. Presence of Heme was measured at different dilutions of the supernatant by reading absorbance at 416 nm using an H1 Synergy Plate Reader to create a negative control.

Blood samples (K2EDTA) for testing hydration capabilities of the different water compositions over time were prepared by diluting the blood with a hypertonic solution to 6% (i.e., the RBCs were shriveled). The three water composition samples were added to respective blood samples, which were then incubated, centrifuged, and the supernatant removed and analyzed for absorbance at 416 nm (to recognize Heme content). Hemolysis analysis was run at 5 minutes, 10 minutes, 30 minutes, and 60 minutes of exposure to the water compositions to determine if hemolysis is dependent upon exposure to the water compositions over time.

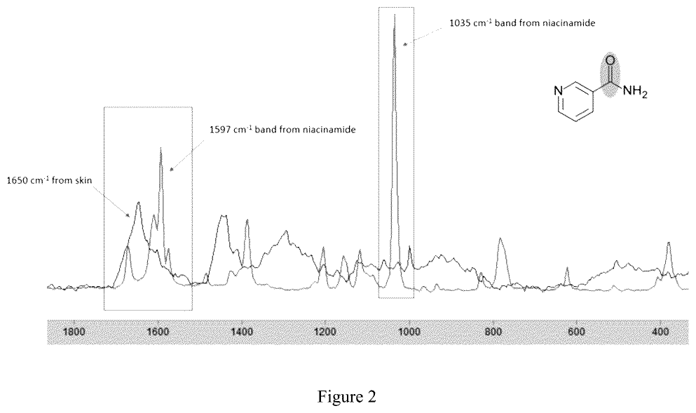

As displayed in FIG. 2, each of the sample compositions demonstrated a time-dependent increase in hydration. However, the RO w/Macro sample (i.e., not prepared in accordance with the present disclosure) had the lowest Heme signal at all time points, demonstrating limited potential for hydration even after an extended period of time. The EDI Tech #2 sample, demonstrated the highest Heme signal at 5 minutes, indicating a strong hydration potential even after only 5 minutes of exposure to the RBCs. The EDI Tech #2 sample also demonstrated a small time-dependent increase in hemolysis, but by 60 minutes the EDI Macro #2 had reached the same hydration potential as the EDI Tech #2 sample. Thus, the EDI Tech #2 was capable of significant hydration even at short durations of exposure.

### Example 3: Beverage Composition Hydration Study

Beverage compositions were prepared by blending 8.1 mg calcium chloride dihydrate, 6.1 mg sodium bicarbonate, 9.0 mg magnesium sulfate heptahydrate, and 2.8 mg zinc sulfate monohydrate into 500 mL conventional purified water (control beverage), or by blending 8.1 mg calcium chloride dihydrate, 6.1 mg sodium bicarbonate, 9.0 mg magnesium sulfate heptahydrate, and 2.8 mg zinc sulfate monohydrate into 500 mL of a composition prepared according to the disclosure (investigational beverage).

Healthy adult male participants were randomly assigned in a double-blind manner to consume the control beverage or the investigational beverage over the course of the study. The degree of hydration in the participants after consuming either the investigational beverage or the control beverage was measured before, during, and after exercise for each of the participants, and a number of hydration-related characteristics were assessed.

Participants who consumed the investigational beverage had an average 1.7% decrease in blood serum osmolality from pre-exercise to two hours post-exercise versus an average 1.1% decrease in the control beverage group. Moreover, a greater proportion of participants in the investigational beverage group (87%) than in the control beverage group (67%) had a decrease in blood serum osmolality at two hours post-exercise.

Participants who consumed the investigational beverage had average increases of 5.1% in total body water (TBW) and 5.6% (p=0.478) in intracellular water (ICW) from pre-to two hours post-exercise. In contrast, the participants who consumed the control beverage has only average increases of 2.1% (p=0.011) TBW and 2.0% (p=0.025) ICW from pre-to two hours post-exercise.

A greater proportion of participants who consumed the investigational beverage had an increase in mean corpuscular volume (MCV) from pre-to immediately post-exercise (40%) compared to the group of participants who consumed the control beverage (20%). Additionally, a greater proportion of participants who consumed the investigational beverage had an increase in mean corpuscular volume (MCV) from pre-to two hours post-exercise (40%) compared to the group of participants who consumed the control beverage (33%).

### Example 4: Beverage Composition Dietary Collagen Absorption and Skin Improvement Study

A single-center, double-blind, randomized, placebo-controlled test was carried out to determine the ability of beverages made with water compositions of the present disclosure to improve dietary collagen absorption and to improve facial skin conditions and appearance. Beverage compositions were prepared by blending marine collagen (active ingredient; sourced from cod fish), maltodextrin, vitamin C, gum acacia, niacinamide, monkfruit extract, and zinc sulfate into conventional purified water (Positive Control Beverage), and into a water composition prepared according to the disclosure (Test Beverage). Marine collagen was added to these two beverage compositions in a concentration of 5 g of marine collagen per 355 mL water. A negative control was also prepared by blending maltodextrin, vitamin C, gum acacia, niacinamide, monkfruit extract, and zinc sulfate into conventional purified water (Placebo Beverage), which lacked the marine collagen (active ingredient) of the Positive Control Beverage and the Test Beverage.

Healthy adult participants (both men and women) with a mean age of 55.1 years and mild to moderate facial fine lines or wrinkles were randomly assigned in a double-blind manner to consume the Placebo Beverage, Positive Control Beverage, or the Test Beverage once per day for a course of 12 weeks. The degree of skin resiliency and skin elasticity of the participants' facial skin (i.e., cheeks) was clinically measured by Cutometer MPA 580 (under the eye on the orbital bone) at baseline before consumption of the beverages and during the testing (at 8 and 12 weeks).

A majority (53.8%) of participants who consumed the Test Beverage demonstrated improvement in skin resiliency and elasticity over the 12-week study based on the Cutometer measurements, while the majority of subjects in both the Placebo Beverage and Positive Control Beverage experienced a worsening of both skin resiliency and elasticity. Of the Placebo Beverage cohort, 74.1% experienced a reduction in skin elasticity, while 77.8% experienced a reduction in skin resiliency. Additionally, of the cohort consuming the Positive Control Beverage, 66.7% experienced a reduction in skin elasticity, while 51.9% experienced a reduction in skin resiliency.

The Test Beverage cohort also demonstrated a statistically significant improvement over the Placebo Beverage cohort in resiliency measurements.

### Example 5: Water Beverage Perceived Temperature and Refreshment Study

In early taste studies with water compositions according to the disclosure herein, it was noted that the temperature of the water was transferred to the oral cavity and the throat in a fashion that was stronger and longer lasting than regular reverse osmosis (RO) water. To better quantify and understand this finding, a controlled experiment was set up to test DI water, RO water, DI water according to the disclosure herein, and RO water according to the disclosure herein to assess this observed effect.

Procedure: All water samples were dispensed into 16-ounce clear plastic labeled bottles. A salt concentrate solution (Table 2) was prepared and 5 ml of the salt concentrate solution was pipetted into each 500 ml bottle of water. The salt concentrate solution was prepared separately for each test water, e.g. DI water, RO water, DI water prepared according to the disclosure herein (Investigative), and RO water prepared according to the disclosure herein (Investigative). The samples were placed in the refrigerator overnight and tasted the next day. Samples were removed from the refrigerator only at the time of tasting to ensure that the bottles were not allowed to warm up. Samples were tasted by two trained panelists. Panelist 1 was a 20+ year trained flavorist and Panelist 2 was a 15+ year flavor R&D leader.

The results demonstrate that the DI water according to the disclosure herein, and RO water according to the disclosure herein both enhance the upfront coolness intensity and duration in the oral cavity and then also enhance and extend the coolness perceived in the throat as a function of the temperature of the beverage, Table 3. As would be expected, the room temperature (Ambient) water samples had less of an impact on perceived cooling, because the water was warmer and would be expected not to trigger the thermosensitive transient receptor potential (TRP) channels as strongly. In contrast, the refrigerated water, being much colder, delivered a lower temperature to the TRP channels in the mouth and throat resulting in a stronger TRP channel activation.

TABLE 2
Salts used to generate 1 L salt concentrate solution,
which is used to adjust hydration
salts in the finished product.

| | Amount (grams into 1 L |
|---|---|
| Name | of the test water) |
| Magnesium Chloride | 1.5 |
| Calcium Chloride | 1.0 |
| Sodium Bicarbonate | 1.0 |

TABLE 3
Tasting results generated by trained tasters
in the food and beverage industry.

| | Panelist 1 | Panelist 2 |
|---|---|---|
| Name | duration | duration |
| DI Control refrigerator | | |
| Mouth cooling | 5 seconds | 6 seconds |
| Throat cooling | 26 seconds | 37 seconds |
| RO (Investigative) | | |
| refrigerator | | |
| Mouth Cooling | 46 seconds | 40 seconds |
| Throat Cooling | 60 seconds | 91 seconds |
| DI (Investigative) | | |
| refrigerator | | |
| Mouth Cooling | 40 seconds | 45 seconds |
| Throat Cooling | 81 seconds | 120 seconds |
| DI Control Ambient | | |
| Mouth cooling | 0 seconds | 10 seconds |
| Throat cooling | 15 seconds | 20 seconds |
| RO Control Ambient | | |
| Mouth cooling | 23 seconds | 10 seconds |
| Throat cooling | 23 seconds | 22 seconds |
| RO (Investigative) Ambient | | |
| Mouth Cooling | 48 seconds | 21 seconds |
| Throat Cooling | 48 seconds | 43 seconds |
| DI (Investigative) Ambient | | |
| Mouth Cooling | 0 seconds | 15 seconds |
| Throat Cooling | 18 seconds | 33 seconds |

### Example 6: Study to Investigate Water Composition Formulations on the Penetration and Absorption of Actives into Human Skin

A study was conducted to determine the impact of specific water-based formulations on the penetration of actives into human skin. The formulations tested used either DI water or water prepared in accordance with the disclosure.

Six water-based formulations were prepared:
1. Formulation 1 containing 3% niacinamide and DI water.
2. Formulation 2 containing 3% niacinamide and water prepared in accordance with the disclosure.
3. Formulation 3 containing bakuchiol and DI water.
4. Formulation 4 containing bakuchiol and water prepared in accordance with the disclosure.
5. Formulation 5 containing Everwhite and DI water.
6. Formulation 6 containing Everwhite and water prepared in accordance with the disclosure.

A control formulation, DI water alone, was also used for comparison.

Experimental protocol: Confocal Raman measurements were carried out on treated human skin samples. First, flash-frozen human skin from the same donor, purchased from a licensed supplier, was obtained (this skin was used for all Raman experiments). 2 cm×2 cm pieces of skin were thawed and cleaned. Then, 40 mg (excess of sample) of each formulation was applied to the skin surface of each piece and massaged with a glass rod for 60 seconds. The skin was then placed at the interface of a Franz diffusion cell with the acceptor chamber filled with DI water. Human skin was purchased from a licensed supplier. The skin samples used in the Raman experiments were all from the same batch.

The opening of the donor cell was covered with parafilm, and the cell was incubated at 32° C. for 2 hours. After 2 hours, any excess formulation on the skin surface was gently blotted with a Kimwipe. The treated skin samples were then incubated prior to Raman spectroscopy analysis. The skin samples were scanned by confocal Raman spectroscopy at 2 months post-treatment (and for the niacinamide samples, also at 18 months post-treatment) to evaluate and compare active absorption/penetration inside the stratum corneum and beyond, as well as the water content of the skin pieces.

### Ex—Vivo Studies of Skin Penetration of Actives:

Confocal Raman spectroscopy was used to visualize the niacinamide, bakuchiol, or Everwhite delivery into the stratum corneum and beyond into the epidermis, as a function of the inclusion of either the Hydrosome or EDI water. All spectra were collected with the following parameters:
Spectral resolution: 4 cm-1
Spectral range: 4000-400 cm-1
Laser excitation: 532 nm
Laser power: 20 mw
Laser exposure: 8 s

For the niacinamide penetration studies, Raman fingerprint region spectra for niacinamide and virgin human skin samples were compared. FIG. 2 depicts an overlaid Raman fingerprint region spectra of niacinamide (red) against virgin skin sample (blue). The strong band contributions at 1035 cm⁻¹, 1597 cm⁻¹ from niacinamide, and the 1650 cm⁻¹ amide I contribution from skin, were used to monitor the niacinamide penetration into the skin. FIG. 2 shows the typical Raman spectrum recorded on the niacinamide (raw material) and on virgin human skin.

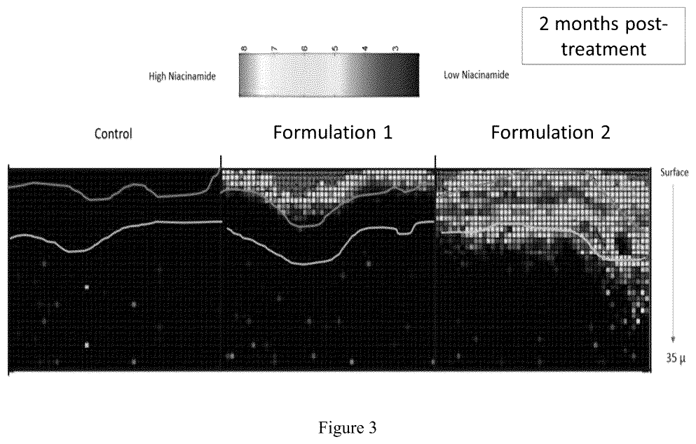

Specific Raman images were generated at 2 months post-treatment to visualize the niacinamide distribution inside the treated skin samples. Hyperspectral images for the control sample, the Formulation 1 sample, and the Formulation 2 sample at 2 months post-treatment show the integrated intensity of 1597 cm⁻¹ normalized to the amide I band at 1650 cm⁻¹ (FIG. 3). The highest niacinamide content is represented as red in the color bar, while the lowest niacinamide content is blue. The blue line represents the surface of the SC, while the green line is the boundary between the SC and the viable epidermis (VE).

The Hyperspectral image displayed in FIG. 3 shows a clear distinction between niacinamide formulation behaviors at 2 months post-treatment. From this image:

There is no niacinamide displayed in the control.

Niacinamide does not appear to enter the stratum corneum in Formulation 1.

Niacinamide penetrates into and appears to traverse and enrich the stratum corneum (potentially traveling slightly beyond stratum corneum into the viable epidermis).

A film of niacinamide appears to have formed at the surface of the stratum corneum in Formulation 2 as well.

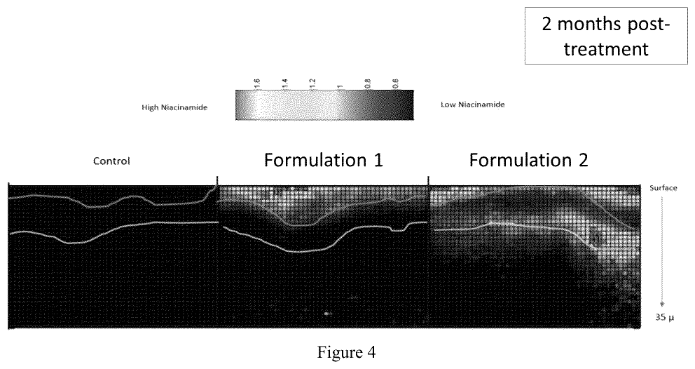

To confirm the FIG. 3 image, the 1035 cm-1 band height is compared to the amide I band height in FIG. 4. Visualizing the niacinamide concentration through the ratioed intensity of the 1035 cm⁻¹ to the amide I largely confirms the observations of FIG. 3. The results indicate that without water prepared in accordance with the disclosure, niacinamide does not enter the stratum corneum. By comparison when formulated with water prepared in accordance with the disclosure, the niacinamide transverses the entire stratum corneum.

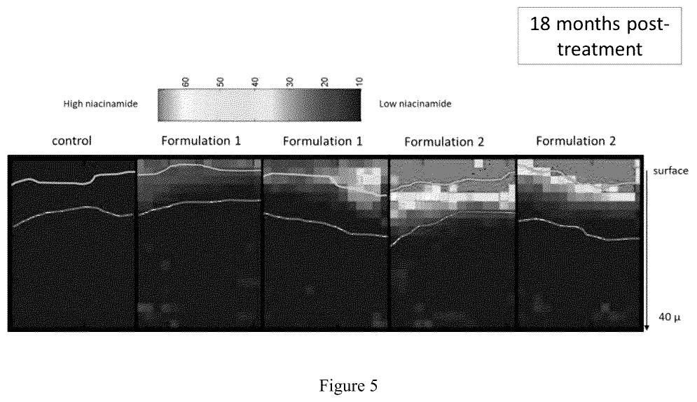

At 18 months post-treatment, the Raman spectroscopy scans for the niacinamide skin samples were repeated. Specific Raman images were generated at 18 months post-treatment to visualize the niacinamide distribution inside the treated skin samples. FIG. 5 shows hyperspectral images of the control skin (no niacinamide, DI water only), Formulation 1 (with 3% niacinamide and DI water), and Formulation 2 (3% niacinamide and water prepared in accordance with the disclosure herein). The image is obtained from the 1597 cm⁻¹ band area normalized to the amide I band at 1650 cm⁻¹. The highest niacinamide content is represented as red in the color bar, while the lowest niacinamide content is blue. The green lines represent the surface of the stratum corneum, while the red lines are the boundary between the stratum corneum and the viable epidermis (VE). The experiments were done in duplicate.

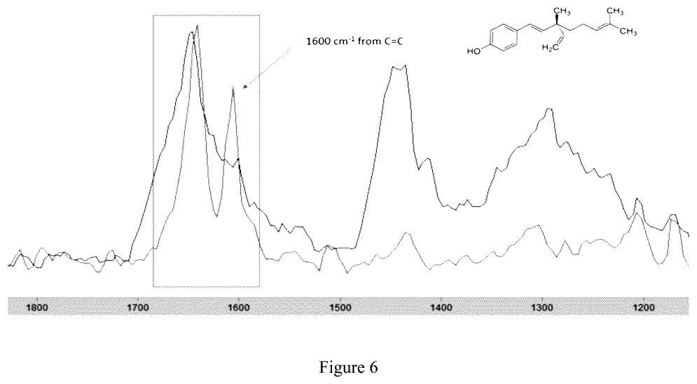

For the bakuchiol penetration studies, Raman fingerprint region spectra for bakuchiol and virgin human skin samples were compared. FIG. 6 depicts an overlaid Raman fingerprint region spectra of bakuchiol (red) against virgin skin sample (blue). The strong band contribution at 1604 cm⁻¹ from bakuchiol is used to monitor its penetration into the skin. Note there is also a strong bakuchiol contribution near the amide 1650 cm⁻¹ amide I band from skin, for this reason normalization is accomplished from the CH3 stretching vibration at 2935 cm⁻¹. The Raman spectrum from bakuchiol has two strong unsaturation bands. The band near 1600 cm⁻¹ is distinct enough from the underlying skin spectrum to be used as a marker band for the determination of bakuchiol penetration in the resulting hyperspectral image.

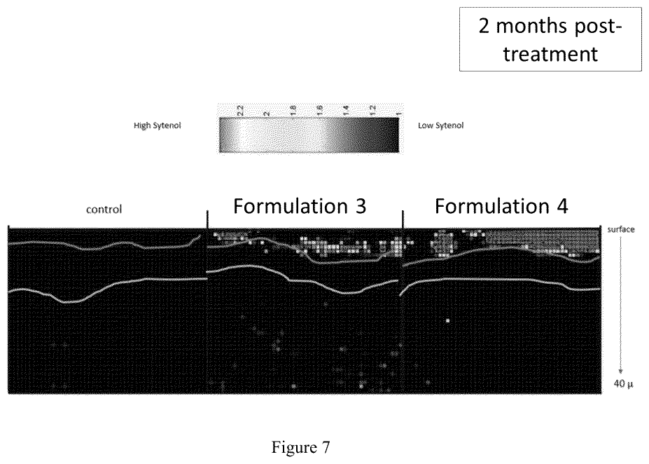

Specific Raman images were generated to visualize the bakuchiol distribution inside the treated skin samples. Hyperspectral images for the control sample, the Formulation 3 sample, and the Formulation 4 sample are depicted in FIG. 7. The image is obtained from the integrated band height of the 1604 cm⁻¹ band normalized with the band at 2935 cm⁻¹. The highest bakuchiol content is represented as red in the color bar, while the lowest bakuchiol content is blue. The blue line represents the surface of the stratum corneum, while the green line is the boundary between the stratum corneum and the viable epidermis.

The Hyperspectral image displayed in FIG. 7 shows a clear distinction between bakuchiol formulation behaviors. As expected, bakuchiol is not found in the control image. Bakuchiol does not penetrate the stratum corneum for either Formulation 3 or 4; however, a more consistent film of bakuchiol appears at the stratum corneum surface for Formulation 4.

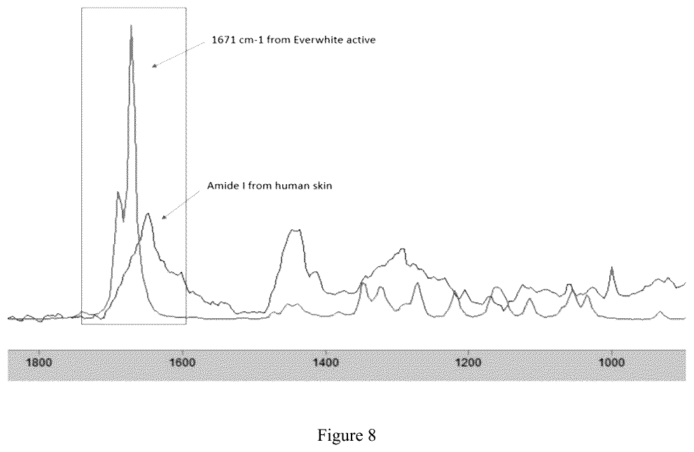

For the Everwhite penetration studies, Raman fingerprint region spectra for Everwhite and virgin human skin samples were compared. FIG. 8 depicts an overlaid Raman fingerprint region spectra of Everwhite (red) against virgin skin sample (blue). The strong band contribution at 1671 cm⁻¹ from the Everwhite active is used to monitor its penetration into the skin. Note there is also a strong Everwhite contribution near the amide 1650 cm⁻¹ amide I band from skin, for this reason normalization is accomplished from the CH3 stretching vibration at 2935 cm⁻¹.

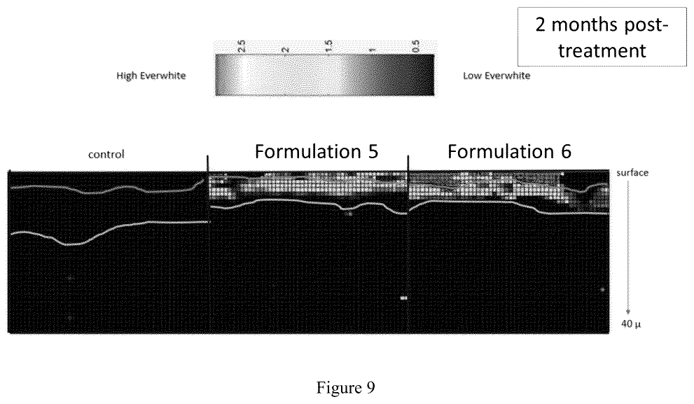

Specific Raman images were generated to visualize the Everwhite active distribution inside the treated skin samples. Hyperspectral images for the control sample, the Formulation 5 sample, and the Formulation 6 sample are depicted in FIG. 9. The image is obtained from the integrated band height of the 1671 cm⁻¹ band normalized with the band at 2935 cm⁻¹. The highest Everwhite content is represented as red in the color bar, while the lowest Everwhite content is blue. The blue line represents the surface of the stratum corneum, while the green line is the boundary between the stratum corneum and the viable epidermis.

The Hyperspectral image displayed in FIG. 9 no Everwhite active is present in the control (as expected). The Everwhite active in both Formulations 5 and 6 penetrated the stratum corneum as marked by the blue boundary layer. The Everwhite active in both Formulations 5 and 6 is contained within the stratum corneum, not penetrating past the stratum corneum/viable epidermis boundary as marked by the green line. Notably, however, Formulation 6 displayed a higher active concentration (film) of the Everwhite active at the surface of the stratum corneum compared to the DI water-containing Formulation 5.

### Ex—Vivo Studies of Water Content in Skin as a Result of Exposure to Formulations:

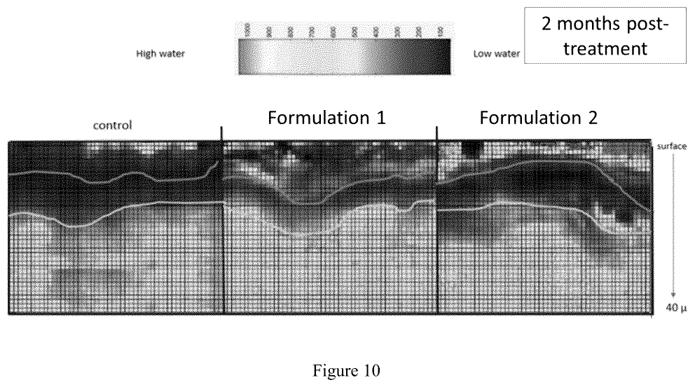

Using a defined portion of the O—H stretching vibration (3350-3550 cm⁻¹) normalized to the amide I band at 1650 cm⁻¹, the water content present within the skin after exposure to each of the formulations (Formulations 1-6) and the Control DI water formulation are compared both visually and quantitatively. These images are captured in FIGS. 10-13.

In FIG. 10, hyperspectral images of the control skin (DI water), Formulation 1 (with 3% niacinamide and DI water), and Formulation 2 (3% niacinamide and water treated in accordance with the disclosure) are displayed. The image is obtained from the integrated area of a portion of the O—H stretch (3350-3550 cm⁻¹) that is normalized to the protein amide I band at 1650 cm⁻¹. The highest water content is represented as red in the color bar, while the lowest water content is blue. The blue line represents the surface of the stratum corneum, while the green line is the boundary between the stratum corneum and the viable epidermis.

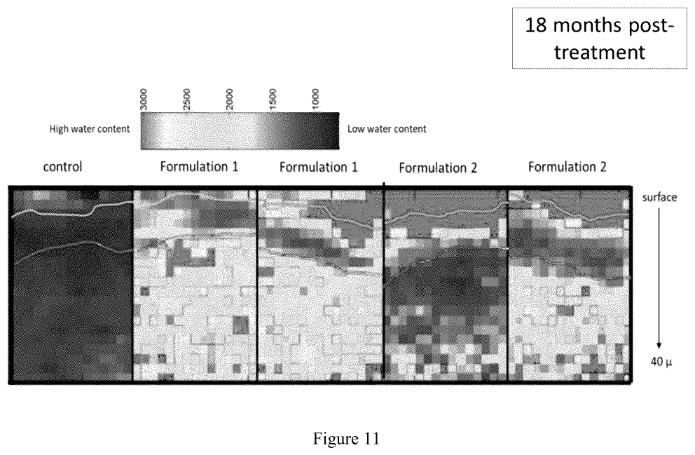

Specific Raman images were also generated at 18 months post-treatment to visualize the water content distribution inside the niacinamide treated skin samples. FIG. 11 shows hyperspectral images of the control skin (no niacinamide, DI water only), Formulation 1 (with 3% niacinamide and DI water), and Formulation 2 (3% niacinamide and water prepared in accordance with the disclosure herein). The image is obtained from the integrated area of a portion of the O—H stretch (3350-3550 cm-1) that is normalized to the protein amide I band at 1650 cm-1. The highest water content is represented as red in the color bar, while the lowest water content is blue. The green lines represent the surface of the stratum corneum, while the red lines are the boundary between the stratum corneum and the viable epidermis (VE). The experiments were done in duplicate.

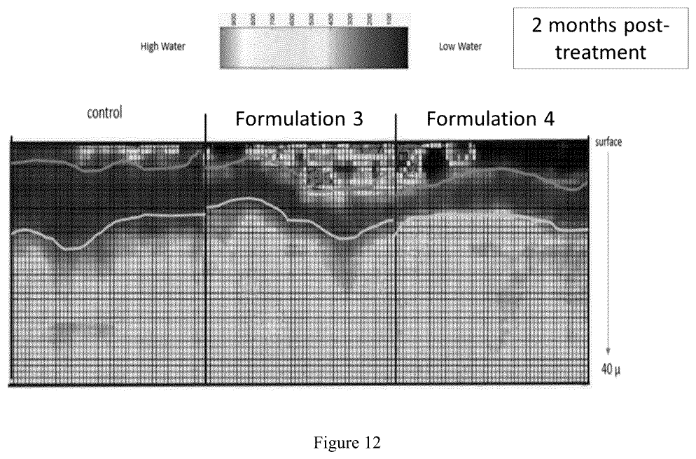

In FIG. 12, hyperspectral images of the control skin (DI water), Formulation 3 (with 2% bakuchiol and DI water), and Formulation 4 (2% bakuchiol and water prepared in accordance with the disclosure) are displayed. The image is obtained from the integrated area of a portion of the O—H stretch (3350-3550 cm⁻¹) that is normalized to the protein amide I band at 1650 cm⁻¹. The highest water content is represented as red in the color bar, while the lowest water content is blue. The blue line represents the surface of the stratum corneum, while the green line is the boundary between the stratum corneum and the viable epidermis.

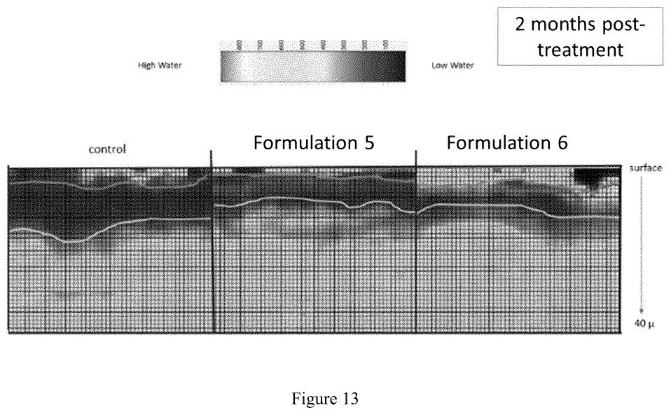

In FIG. 13, hyperspectral images of the control skin (DI water), Formulation 5 (with 3% Everwhite and DI water), and Formulation 6 (3% Everwhite and water treated in accordance with the disclosure) are displayed. The image is obtained from the integrated area of a portion of the O—H stretch (3350-3550 cm⁻¹) that is normalized to the protein amide I band at 1650 cm⁻¹. The highest water content is represented as red in the color bar, while the lowest water content is blue. The blue line represents the surface of the stratum corneum, while the green line is the boundary between the stratum corneum and the viable epidermis.

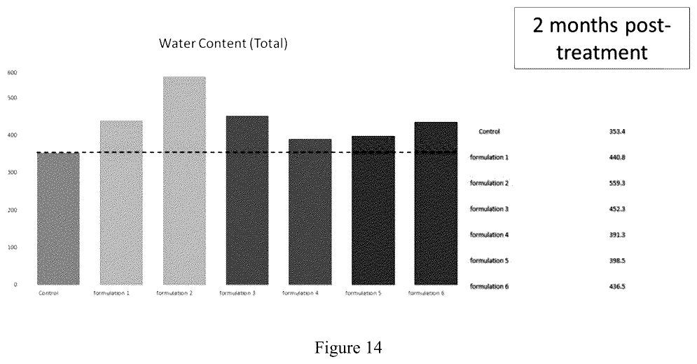

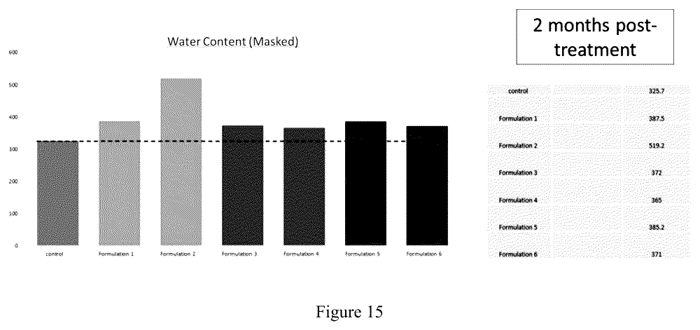

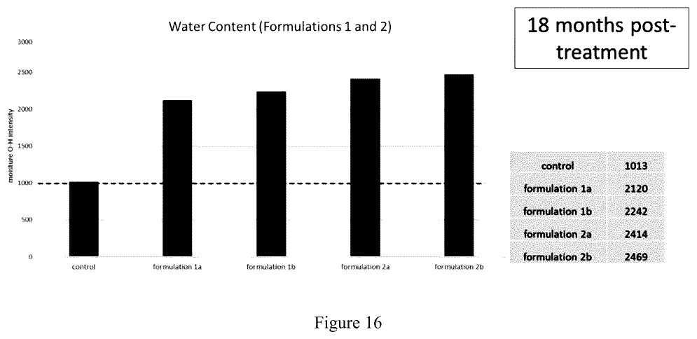

FIGS. 14-16 display the quantitative results from FIGS. 10-13. FIG. 14 displays the total water content from each hyperspectral image for each of Formulations 1-6 (in comparison to Control). In FIG. 14, quantitative hydration results from the hyperspectral images from Formulations 1-6 are shown. Numerical values are obtained from the integrated area of the O—H stretching vibration between 3350-3550 cm⁻¹ normalized to the amide I band. FIG. 15 displays the water content after the area above the stratum corneum has been masked to mitigate excess formulation or film above the skin that may skew the results internal to the skin for each of the Formulations 1-6 and the Control. FIG. 16 displays quantitative hydration results from the hyperspectral images from Formulations 1 and 2 in comparison with the Control from the niacinamide-treated samples 18 months post-treatment where the area above the stratum corneum has been masked to mitigate excess formulation or film above the skin that may skew the results internal to the skin.

FIGS. 14-16 demonstrate that the lowest amount of hydration is observed in the Control sample, while Formulation 2 (niacinamide with water prepared in accordance with the disclosure) clearly provides the highest degree of hydration (and maintains this higher degree of penetration).

### Example 7: Demonstration of Enhanced Skin Permeation at Two Years

The present study evaluates the penetration enhancement of niacinamide in ex-vivo human skin using two formulations: a control preparation comprising deionized (DI) water and a test preparation containing an ultrafine bubble suspension known as Ultrafine bubble suspension. The objective was to assess whether the Ultrafine bubble suspension formulation could enhance the delivery of niacinamide into the stratum corneum (SC) and the viable epidermis (VE) compared to the DI water formulation.

The experiment utilized confocal Raman spectroscopy to monitor the penetration of niacinamide in ex-vivo human skin samples. Two formulations were tested:
Control: 3% niacinamide in DI water.
Test: 3% niacinamide in Ultrafine bubble suspension.

Human skin samples were flash-frozen, thawed, and treated with either the control or test formulation. The skin was then placed in a Franz diffusion cell and incubated at 32° C. for two hours. Post-incubation, the skin surface was blotted, and the niacinamide penetration into the SC and VE was analyzed using confocal Raman spectroscopy.

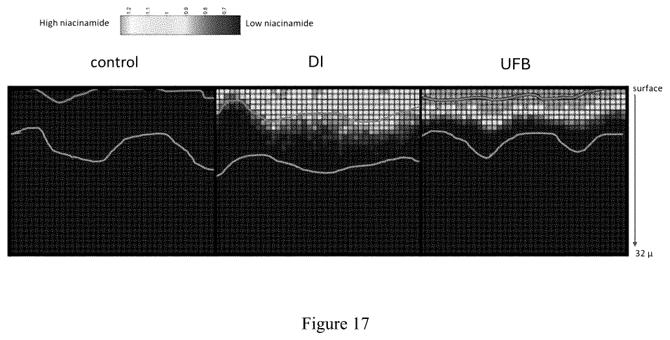

The results demonstrated a significant difference in niacinamide penetration between the control and test formulations:
Control (DI Water): Niacinamide remained primarily on the skin surface with minimal penetration into the SC. No niacinamide was detected in the VE.
Test (Ultrafine bubble suspension): Niacinamide exhibited deeper penetration into the SC with a higher concentration on the skin surface. Although the penetration did not extend into the VE, the formulation with Ultrafine bubble suspension showed a more consistent and robust deposition of niacinamide at the surface. (FIG. 17).

These findings suggest that the inclusion of Ultrafine bubble suspension in topical formulations can significantly enhance the penetration and surface deposition of active ingredients like niacinamide, potentially leading to more effective skincare products.

### Example 8: Study to Investigate Water Composition Formulations for Efficacy in Antibacterial Solutions Against Staphylococcus aureus

Test substances for assessing antibacterial activity against S. aureus bacteria were prepared as follows:
DI Water (comparative control)
Prepared Water (investigative control)
DI Water with 0.25% Phenoxyethanol
DI Water with 0.5% Phenoxyethanol
DI Water with 1.0% Phenoxyethanol
Prepared Water with 0.25% Phenoxyethanol
Prepared Water with 0.5% Phenoxyethanol
Prepared Water with 1.0% Phenoxyethanol

Prior to test initiation, 10.0 mL of each test substance were aliquoted into individual sterile conical tubes. Additionally, 10.0 mL of sterile Phosphate Buffered Saline (PBS) were aliquoted into individual sterile conical tubes to be used for the microbial population controls. The number of replicates and test microorganisms was considered to determine the number of sterile conical tubes to prepare.

Test cultures for the test microorganism were initiated in Tryptic Soy Broth (TSB) and allowed to incubate under conditions necessary for sufficient growth of S. aureus (see Table 4). The test inoculum was prepared from the test culture by centrifuging and resuspending in an appropriate volume of PBS and then diluting the test culture in PBS to reach the target concentration of ≥1.0×10⁶ CFU/mL.

TABLE 4
Test microorganism S. aureus
growth medium and incubation conditions

| | | Incubation |
|---|---|---|
| Microorganisms | Growth Medium | Conditions |
| Staphylococcus aureus | Tryptic Soy Broth (TSB)/ | 36 ± 1° C. |
| ATCC 6538 | Tryptic Soy Agar (TSA) | Aerobic |

The individual test substance aliquots were then each inoculated with 0.100 mL of the S. aureus test microorganism. A calibrated digital timer was started at the time of inoculation to ensure each aliquot met the appropriate contact time. The inoculated test substances were vortex mixed and allowed to dwell for the remainder of the contact time. Following the completion of the contact time, each inoculated suspension was neutralized by transferring 1.000 mL of the suspension to 9.0 mL of sterile D/E Broth. The neutralized suspensions were each enumerated by 1:10 serial dilutions in sterile PBS.

The microbial population control, i.e. Time Zero, was used to determine the concentration of the inoculum for each test microorganism. The individual control substance aliquots were each inoculated with 0.100 mL of the target test microorganism and vortex mixed. Following inoculation, the inoculated time zero suspensions were immediately neutralized after mixing by transferring 1.000 mL of the suspension to 9.0 mL of sterile D/E Broth. The neutralized suspensions were each enumerated by 1:10 serial dilutions in sterile PBS.

For contact times greater than 1 hour, a Time Final concentration was used to confirm the viability of the microorganism for the duration of the test. The individual control substance aliquots were each inoculated with 0.100 mL of the S. aureus and vortex mixed. Following inoculation, the inoculated time final suspensions were immediately neutralized after mixing by transferring 1.000 mL of the suspension to 9.0 mL of sterile D/E Broth. The neutralized suspensions were each enumerated by 1:10 serial dilutions in sterile PBS.

Percentage reduction and Log₁₀ reduction in the S. aureus microorganisms were observed (Table 5).

TABLE 5
S. aureus percent reduction and Log₁₀ reduction at 24 hours compared to Time Zero.

| | | | | | Percent | Log₁₀ |
|---|---|---|---|---|---|---|
| | | | | | Reduction | Reduction |
| | | | | | Compared | Compared |
| | Test | | Contact | | to Time | to Time |
| Test Date | Microorganism | Test Substance | Time | CFU/mL | Zero | Zero |
| 25 APR. 2023 | S. Aureus | CONTROL | Time Zero | 1.15E+07 | N/A | N/A |
| 26 APR. 2023 | ATCC 6538 | DI Water | 24 Hours | 1.11E+07 | 3.06% | 0.01 |
| | | Prepared Water | | 9.50E+06 | 17.03% | 0.08 |
| | | DI Water with | | 1.26E+07 | None | None |
| | | 0.25% | | | | |
| | | Phenoxyethanol | | | | |
| | | DI Water with | | 1.10E+07 | 4.37% | 0.02 |
| | | 0.5% | | | | |
| | | Phenoxyethanol | | | | |
| | | DI Water with | | 6.50E+03 | 99.94% | 3.25 |
| | | 1.0% | | | | |
| | | Phenoxyethanol | | | | |
| | | Prepared Water | | 1.09E+07 | 5.24% | 0.02 |
| | | with 0.25% | | | | |
| | | Phenoxyethanol | | | | |
| | | Prepared Water | | 1.18E+07 | None | None |
| | | with 0.5% | | | | |
| | | Phenoxyethanol | | | | |
| | | Prepared Water | | 3.10E+02 | 99.997% | 4.57 |
| | | with 1.0% | | | | |
| | | Phenoxyethanol | | | | |

The neutralization verification (NV) tests were performed for DI water w/1.0% Phenoxyethanol and Prepared water w/1.0% Phenoxyethanol on the test microorganisms. The test microorganism was diluted in sterile PBS, targeting a final concentration of 10-100 CFU/mL, and was used as the neutralization verification inoculum.

Notably, S. aureus percent reduction and Log₁₀ reduction were significantly higher in the Prepared water investigative control (i.e., without antibacterial agent phenoxyethanol) than in the DI water comparative control. The result suggests that the prepared water composition alone may serve as an effective antibacterial agent. Additionally, while the test composition including DI water with 0.25% phenoxyethanol exhibited no percentage or logarithmic reduction in S. aureus after 24 hours, the composition including 0.25% phenoxyethanol and Prepared water exhibited higher efficacy (i.e., 5.24% reduction and a 0.02 Log₁₀ reduction compared to Time Zero). The composition of Prepared water/1.0% phenoxyethanol also outperformed its DI water counterpart, with a 99.997% reduction and a 4.57 Log₁₀ reduction compared to the DI water/1.0% phenoxyethanol composition's 99.94% reduction and 3.25 Log₁₀ reduction. This further suggests that water compositions prepared in accordance with the disclosure may increase the efficacy of antibacterial agents.

### Example 9: Effects of Ultrafine Bubbles on Gut Microbiome

The described rodent study evaluates the effects of drinking ultrafine bubble (UFB) suspensions on the gut microbiome and overall health. This study offers compelling insights into the biological impact of UFB water, specifically on gut microbial composition, inflammatory response, and metabolic parameters, which can support claims for the beneficial effects of UFB products on consumer health.

### Summary of Experiment

The study, titled “To Evaluate the Modulation of Gut Microbiota in Healthy Rats after Exposure to ultrafine bubble (UFB) water, and Deionized Water (DI)”, involved 24 female Sprague Dawley rats divided into two groups. Group 1 (G1) received deionized water (DI), and Group 2 (G2) received ultrafine bubble (UFB) water for 12 consecutive weeks via oral administration ad libitum. Metagenomic, biochemical, and histopathological analyses were performed at Weeks 0, 6, 8, and 12 to assess changes in gut microbiota, clinical pathology, general health markers (triglyceride and blood glucose levels, liver enzymes, inflammatory markers) and short-chain fatty acids (SCFA).

The study revealed no statistically significant differences in body weight, feed consumption, or water intake between the two groups. However, natural fluctuations in water consumption were observed throughout the experimental period, likely due to biological factors such as metabolism and estrous cycles in the rats. Notably, both groups showed a significant shift in gut microbial composition over the study. Initially, the microbiota was dominated by the phyla Bacteroidetes and Firmicutes. By Weeks 8 and 12, a marked change in the balance between these two phyla was observed in both groups. This shift was more pronounced in ultrafine bubble (UFB) suspension group, resulting in a significantly different Firmicutes to Bacteroidetes ratio between the two groups.

Short-chain fatty acids (SCFAs) were detected in both groups with a significant increase at week 8 and week 12. The ultrafine bubble (UFB) water group exhibited statistically higher concentrations of iso-valerate, valerate at week 8 and butyrate and valerate at week 12. The presence of these SCFAs in the ultrafine bubble (UFB) water group suggests that UFB water can enhance SCFA production. This is particularly relevant, as SCFAs play a key role in maintaining gut health by supporting gut integrity and exerting anti-inflammatory effects.

In terms of inflammatory markers, the ultrafine bubble (UFB) water group showed lower levels of key cytokines, including IL-1β, TNF-α, and IL-10, compared to the DI control group, especially at Week 12. This trend suggests that UFB water may have anti-inflammatory properties, further contributing to overall gut health improvements. Collectively, these results support the potential for UFB suspensions to modulate gut microbiota composition, enhance SCFA production, and reduce inflammation, indicating a range of health benefits for consumers

### Example 10: Effects of Ultrafine Bubbles on Water Sparged with Nitrous Oxide

The experiment described below investigates the effects of generating ultrafine bubbles in water sparged with a gas other than air, such as nitrous oxide (N₂O), specifically focusing on the impact on taste. This experiment demonstrates several key points: 1) ultrafine bubble composition can be tailored through sparging and/or microbubble enrichment; 2) the presence of ultrafine bubbles can improve the stability and shelf-life of dissolved gases in water; and 3) there are potential applications for flavor enhancement and taste improvements related to both effects. Summary of Experiment

This experiment investigated the effectiveness of nanobubble generation technology in enhancing N₂O infusion into water. Two identical BrewBuilt fermenters were used—one as a test vessel with an ultrafine bubble generator installed in a recirculating loop and the other as a control with a recirculating loop passing through a valve to ensure identical flow rates—each receiving equal volumes of chilled water and amounts of nitrous oxide. Operating conditions, such as recirculation flow rate, temperature, and pressure, were carefully controlled to minimize observable differences in N₂O absorption between the two samples. Both fermenters were initially chilled, sealed, and recirculated to ensure uniformity before baseline gas measurements were taken. The test fermenter was then infused with N₂O, while the control fermenter received an equivalent gas volume without nanobubble generation. After gas injection, samples were collected and bottled for later evaluation.

Early anecdotal evidence suggested that the samples produced containing ultrafine bubbles, exhibited significantly enhanced sweetness. While both the test and control samples were expected to have some sweetness due to the common effect of N₂O saturation in water and other liquids, the test samples were observed to be perceptibly sweeter than the control samples, despite both receiving the same amount of N₂O under identical conditions—except for the presence of ultrafine bubbles in the test samples. This observation led to conducting a triangle sensory discrimination test. In a triangle test, panelists are given three samples—two identical and one different—and asked to identify the unique sample without prior knowledge of its identity. The test involved three panelists, each tested in triplicate, resulting in a total of nine tests conducted once immediately after opening the bottled samples (one week after production), and another nine tests carried out one hour after opening the bottles, which were kept open under constant temperature conditions. After identifying the sample they believed was different, the panelists were then asked whether the outlier was sweeter or less sweet compared to the others.

The results, shown in tables below, present the outcomes of the triangle test conducted immediately and one hour after opening the bottles containing nitrous oxide-sparged water (CTL) and water sparged with N₂O containing ultrafine bubbles (HW). The panelists were able to distinguish between the two samples 88.9% of the time immediately after opening and 66.7% one hour later, demonstrating a statistically significant difference in sweetness. Whenever the panelists correctly identified the different sample, they consistently noted that the HW samples were sweeter.

TABLE 6
Comparison of beverages containing nitrous oxide-sparged water (CTL)
and water sparged with N2O containing ultrafine bubbles (HW)

| | O hr | | | | | | | | |
|---|---|---|---|---|---|---|---|---|---|
| | Test 1 | | | Test 2 | | | Test 3 | | |
| | HW | HW | CTL | HW | CTL | CTL | HW | CTL | HW |
| Panelist 1 | | X | | | | X | | | X |
| Panelist 2 | | X | | | X | | | | X |
| Panelist 3 | | X | | | X | | | | X |

| Test | | | | |
|---|---|---|---|---|
| 1 | 2 | 3 | Percent Correct | Significance |
| Correct | Wrong | Correct | 88.9% | 0.1% |
| Correct | Correct | Correct | | |
| Correct | Correct | Correct | | |

| | 1 hr | | | | | | | | |
|---|---|---|---|---|---|---|---|---|---|
| | Test 1 | | | Test 2 | | | Test 3 | | |
| | HW | HW | CTL | HW | CTL | CTL | HW | CTL | HW |
| Panelist 1 | | | X | X | | | | | X |
| Panelist 2 | X | | | X | | | | X | |
| Panelist 3 | X | | | X | | | | X | |

| Test | | | | |
|---|---|---|---|---|
| 1 | 2 | 3 | Percent Correct | Significance |
| Correct | Correct | Wrong | 66.7% | 5.0% |
| Wrong | Correct | Correct | | |
| Wrong | Correct | Correct | | |

Without being bound by theory, there are several possible explanations for the results of this experiment: 1) the ultrafine bubbles in the sample were composed of N₂O, which directly contributed to enhanced sweetness and stability; 2) the formation of ultrafine bubbles of any composition facilitate improved dissolution and stability of gaseous solutes in ultrafine bubble suspensions; and/or 3) the presence of ultrafine bubbles enhance the sensory perception, penetration, and/or delivery of gaseous solutes.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims.

What is claimed is:

1. A composition for oral administration, ingestion, or topical application, the composition comprising:
water; and
ultrafine bubbles comprising gases released from solution in the water,
wherein the composition is capable of enhanced delivery of bioactive agents, hydration into mammalian cells, or sensory effects.

2. The composition of claim 1, further comprising:
at least one solute, wherein the at least one solute comprises one or more flavoring agents, sweeteners, anti-acne agents, antibacterial agents, fluoride sources, nutrients, electrolytes, minerals, warming-sensation agents, and cooling-sensation agents.

3. The composition of claim 1, wherein the at least one solute is dissolved within, surrounded by, or stabilized by the ultrafine bubbles.

4. The composition of claim 1, wherein the composition increases cell permeability or bioavailability of the at least one dissolved solute.

5. The composition of claim 1, wherein the ultrafine bubbles have a median diameter of between 2-400 nanometers.

6. The composition of claim 2, wherein the at least one solute is present at a concentration of 0.1-30% by weight of the composition.

7. The composition of claim 1, wherein the ultrafine bubbles remain stable within the composition for at least six months.

8. The composition of claim 2, wherein the at least one solute is stable within the composition for at least six months.

9. The composition of claim 1, wherein the ultrafine bubbles are concentrated within the composition via rotary evaporation, cross flow filtration, or a combination of rotary evaporation and cross flow filtration.

10. The composition of claim 1, wherein the composition is a beverage for ingestion.

11. The composition of claim 2, wherein the at least one solute comprises one or more electrolytes and minerals.

12. A method for enhancing, increasing, and/or retaining hydration in a subject, the method comprising the steps of:
providing the composition of claim 1; and
ingesting the composition by a subject.

13. A method for enhancing absorption of, bioavailability of, or both absorption of and bioavailability of at least one solute in a subject, the method comprising the steps of:
providing the composition of claim 2; and
ingesting the composition by a subject.

14. A method for enhancing absorption of, bioavailability of, or both absorption of and bioavailability of dietary collagen in a subject, the method comprising the steps of:
providing the composition of claim 2 wherein the solute is dietary collagen;
ingesting the composition by a subject.

15. A method of maintaining gut health in a mammal, the method comprising administering the composition according to claim 1 into the gut microbiome of a mammal.

16. The method of claim 15, wherein the composition is capable of modulating gut microbiota composition of the gut microbiome of the mammal.

17. The method of claim 15, wherein the composition is capable of enhancing short chain fatty acid production within the gut microbiome of the mammal.

18. A method for enhancing absorption of, bioavailability of, or both absorption of and bioavailability of a gaseous solute in a subjection, the method comprising the steps of:
providing the composition of claim 2 wherein the solute is a gaseous solute;
administering the composition to a subject.

19. The method of claim 18 wherein the gaseous solute provides enhanced sensor effects of the gaseous solute when ingested by the subject.

20. The method of claim 18 wherein the gaseous solute enhances the sweetness of the composition.

21. The method of claim 18 wherein the gaseous solute is nitrous oxide.

22. A composition for topical application or use, the composition comprising:
water;
ultrafine bubbles comprising gases released from solution in the water; and,
at least one solute, wherein the at least one solute comprises one or more cooling-sensation agents, pain relief agents, numbing agents, anti-acne agents, warming sensation agents, hair restorative agents, antibacterial agents, antiseptic agents, and anti-itch agents.

23. The composition of claim 22, wherein the at least one solute is dissolved within, surrounded by, and/or stabilized by the ultrafine bubbles.

24. The composition of claim 22, wherein the composition increases cell permeability and/or bioavailability of the at least one solute.

25. The composition of claim 22, wherein the at least one solute is at least one of niacinamide, bakuchiol, retinol, 3-O-ethyl ascorbic acid, or combinations thereof.

26. A method of increasing at least one solute absorption into skin cells of a subject, the method comprising topically administering the composition according to claim 25 to intact skin of a subject.

27. The method of claim 26, wherein the solute comprising niacinamide, bakuchiol, retinol, 3-O-ethyl ascorbic acid, or combinations thereof, is absorbed into a stratum corneum layer of the intact skin of the subject.
